(19) United States
(12) Patent Application Publication
(10) Pub. No.: US 2026/0248913 A1
(43) Pub. Date: Aug. 27, 2026

(54) COMPOSITIONS AND METHODS RELATED TO MULTIVALENT BINDERS FOR ANTIVIRAL THERAPEUTICS

(71) Applicants: Carter Swanson, Scottsdale, AZ (US); Arizona Board of Regents on behalf of Arizona State University, Scottsdale, AZ (US)

(72) Inventors: Rizal F. Hariadi, Scottsdale, AZ (US); Carter Swanson, Scottsdale, AZ (US); Swechchha Pradhan, Scottsdale, AZ (US)

(21) Appl. No.: 18/992,379

(22) PCT Filed: Jul. 12, 2023
(86) PCT No.: PCT/US2023/070014

§ 371 Date: Jan. 8, 2025

Related U.S. Application Data
(60) Provisional application No. 63/368,313, filed on Jul. 13, 2022.

(51) Int. Cl. A61K 39/42; A61K 47/54; A61P 31/12; C12N 15/115

(52) U.S. Cl. CPC A61K 39/42; A61K 47/549; A61P 31/12; C12N 15/115; C12N 2310/16

(57) ABSTRACT

The present disclosure provides compositions and methods related to antiviral therapeutics. In particular, the present disclosure provides a highly modular platform useful for the development and production of multivalent biomolecules capable targeting and neutralizing a virus.

### CROSS REFERENCE TO RELATED APPLICATIONS

This application claims priority to and the benefit of U.S. Provisional Patent Application No. 63/368,313 filed Jul. 13, 2022, which is incorporated herein by reference in its entirety for all purposes.

### STATEMENT REGARDING FEDERAL FUNDING

This invention was made with government support under AI144247 awarded by the National Institutes of Health and 2027215 awarded by the National Science Foundation. The government has certain rights in the invention.

### INCORPORATION-BY-REFERENCE OF MATERIAL SUBMITTED ELECTRONICALLY

The text of the computer readable sequence listing filed herewith, titled “40961_601_SequenceListing” created Jul. 11, 2023, having a file size of 186,288 bytes, is hereby incorporated by reference in its entirety.

### FIELD

The present disclosure provides compositions and methods related to antiviral therapeutics. In particular, the present disclosure provides a highly modular platform useful for the development and production of multivalent biomolecules capable targeting and neutralizing a virus.

### BACKGROUND

Antiviral therapy is one of the most exciting aspects of virology, since it has successfully employed basic science to generate very effective treatments for serious viral infections. For example, therapy for human immunodeficiency virus (HIV) infection has demonstrated the potential impact antivirals can have on a lethal, chronic infection with lifesaving therapy administered to millions of individuals. These types of advances are being applied to the treatment of many other viral diseases, such as hepatitis C virus (HCV) infection. The development of new antiviral drugs is very much still in progress, with active drug discovery programs being established for filoviruses, coronaviruses, dengue, and others. Additionally, the conceptual approach to drug development is in flux. In the past, the primary focus has been upon virus targets, and this continues to be a very productive strategy. It is now being complemented by a wider set of approaches, including, for example, compounds that target generic viral targets such as RNA or DNA synthesis and could be active against a range of different viruses and compounds that are directed against host cellular activities necessary for virus replication, which might target one or a spectrum of viruses. Furthermore, established methodologies for drug discovery are now supplemented with the use of large databases and evolving methods in computational biology. However, despite these advances there remains a need for alternative approaches for developing effective antiviral therapies.

### SUMMARY

Embodiments of the present disclosure include a multivalent antiviral biomolecule. In accordance with these embodiments, the biomolecule includes (i) a nucleic acid-based scaffold; and (ii) a plurality of antiviral binders conjugated to the scaffold, wherein each antiviral binder of the plurality of binder targets one or more viral proteins, thereby neutralizing one or more viruses.

In some embodiments, the plurality of antiviral binders target the same viral protein. In some embodiments, the plurality of antiviral binders target two or more distinct viral proteins.

In some embodiments, the nucleic acid-based scaffold comprises a DNA origami-based nanostructure. In some embodiments, the nucleic acid-based scaffold comprises a DNA snub cube. In some embodiments, the DNA snub cube comprises 24 vertices and 60 edges. In some embodiments, at least one edge of the DNA snub cube is conjugated to at least one antiviral binder of the plurality of antiviral binders.

In other embodiments, the scaffold is comprised of non-nucleic acid-based compositions. In some embodiments, the scaffold comprises a polystyrene bead, a quantum dot, and/or a peptide or polypeptide.

In some embodiments, the plurality of antiviral binders are conjugated to the nucleic acid-based scaffold via a linker. In some embodiments, the linker comprises a single-stranded DNA overhang coupled to the scaffold. The single-stranded DNA overhang coupled to the scaffold is also referred to herein as a “handle”. In some embodiments, the linker comprises a single-stranded DNA complementary to the single-stranded DNA overhang. In some embodiments, the complementary single-stranded DNA is conjugated to at least one antiviral binder of the plurality of antiviral binders. In some embodiments, the complementary single-stranded DNA is conjugated to at least one antiviral binder of the plurality of antiviral binders via a SNAP-tag with a benzylguanine (BG) linker.

In some embodiments, at least one of the plurality of antiviral binders comprises a monoclonal antibody, a human antibody, a humanized antibody, a chimeric antibody, and/or a single-domain antibody. In some embodiments, at least one of the plurality of antiviral binders comprises an antibody fragment selected from the group consisting of Fab, Fab-C, Fab′-SH, Fv, scFv, and (Fab′)₂ fragments. In some embodiments, at least one of the plurality of antiviral binders comprises a single-domain antibody (nanobody).

In some embodiments, at least one of the plurality of antiviral binders comprises an aptamer. In some embodiments, the aptamer is an RNA or DNA aptamer. In some embodiments, the aptamer comprises a docking domain to facilitate conjugation to the scaffold. In some embodiments, the aptamer further comprises a linker region between the docking domain and a region of the aptamer that binds a viral protein. In some embodiments, the linker region is from about 2 to about 40 base pairs in length.

In some embodiments, at least about 8 antiviral binders are conjugated to the scaffold. In some embodiments, at least about 12 antiviral binders are conjugated to the scaffold. In some embodiments, at least about 24 antiviral binders are conjugated to the scaffold. In some embodiments, at least about 36 antiviral binders are conjugated to the scaffold. In some embodiments, about 60 antiviral binders are conjugated to the scaffold.

Embodiments of the present disclosure also include a composition comprising a plurality of any of the antiviral biomolecules described herein. In some embodiments, the composition includes a pharmaceutically acceptable adjuvant, excipient, and/or carrier, and wherein the composition is suitable for administration to a subject in need thereof.

Embodiments of the present disclosure also include a method of detecting a target virus using any of the antiviral biomolecules described herein.

Embodiments of the present disclosure also include a method of neutralizing a target virus using any of the antiviral biomolecules described herein.

Embodiments of the present disclosure also include a kit comprising any of the biomolecules described herein, and instructions for detecting or neutralizing a target virus.

### BRIEF DESCRIPTION OF THE DRAWINGS

FIGS. 1A-1B: Representative diagrams illustrating key features of the multivalent antiviral platform of the present disclosure. As described further herein, the multivalent antiviral biomolecules of the present disclosure can be described using the following terms: SC refers to DNA snub cube embodiment; SC60 or SC60H generally refers to a snub cube and the number of conjugation sites/handles; SC60-nbGFP generally refers to a snub cube that comprises a concatenated scaffold and conjugated antiviral binders; nbGFP generally refers to an anti-GFP nanobody, fusion protein with SNAP-tag and BG DNA; aptGFP generally refers to an anti-GFP RNA aptamer with a docking domain.

FIGS. 2A-2B: Representative images using ONI microscopy. Schematic workflow of the assay (FIG. 2A). Representative ONI microscopy images showing binding between nbGFP and PRV 486 (FIG. 2B). Anti-GFP antibody (AbGFP) used as the positive controls for nbGFP and EGFP is used as positive control for PRV 486. Casein (blocking agent) is used as negative control for nbGFP. Scale bar is 10 um.

FIGS. 3A-3C: Design, synthesis, and characterization of DNA origami and nanobody chimera. Schematic showing the stepwise formation of SC-nbGFP using DNA origami technology (FIG. 3A). The technique involves folding the M13mp18 ssDNA using short staple strands in the presence of cations such as Mg²⁺. The 3D rod structure of the snub cube (SC) with 24 vertices and 60 edges is shown on the right. The second step is the incorporation of the BG-DNA bifunctional linkers onto the SC (front face only), followed by the multivalent conjugation of nbGFP virus binders. Agarose gel electrophoresis showing the successive formation of SC60H, SC60H-BG-DNA, and SC60H-nbGFP (FIG. 3B). The unfolded M13mp18 strand is used as a reference. Transmission electron microscopy (TEM) characterization of SC60H showing the wireframe nanostructures (FIG. 3C). The scale bar is 100 nm.

FIGS. 4A-4C: Multivalent presentation of nbGFP on DNA origami scaffold increases viral binding. Schematic of ELISA assay (FIG. 4A). ELISA binding curves of PRV 486 to nbGFP compared to a monoclonal antibody (FIG. 4B). ELISA binding curves of PRV 486 to SC-nbGFP of different valences (FIG. 4C). Absorbance values resulting from non-specific interactions without the virus or EGFP were subtracted. The relative binding affinities of SC1H-nbGFP, SC12H-nbGFP, and SC60H-nbGFP calculated by non-linear regression are listed in the table (on right).

FIGS. 5A-5B: Representative in vitro inhibition data of PRV 486 by SC60H-nbGFP. Schematic of the in vitro experiment (FIG. 5A). Comparison of residual infectivity of PRV 486 after treatment with different groups (FIG. 5B). Data are presented as mean±SEM, N=3 biologically independent experiments. A two-tailed t-test was performed to test significance against the PRV only group (*P<0.05; **P<0.005). The infectivity of PRV only group is used as reference for calculating percent infectivity.

FIGS. 6A-6B: Representative dose response curves. Dose-dependent, plaque-reducing inhibition curves for the SC60-nbGFP (black circle), SC60 (scaffold only; orange square) and nbGFP (binder only, blue triangle) (FIG. 6A). N=2 biologically independent experiments. Representative plaque assays corresponding to the maximum concentration of SC60-nbGFP, SC60 and nbGFP at 150 nM (FIG. 6B).

FIGS. 7A-7B: Representative data using standard design principles of DNA origami in which the number of ssDNA conjugation sites can be designed (FIG. 7A). Conjugation of varying amounts of nanobody (nb) per snub cube is evaluated in gel shift assay (FIG. 7B). Four valencies are evaluated, 1, 2, 3 and 4. For each, the snub cube with corresponding valency along (nH) is run next to two repeats when conjugated nanobody. Conjugated snub cubes with valencies of 8, 12, 24, 36 and 60 are run next to each other. Gel shifts are observed to generally correlate with the degree of valency (FIG. 7B).

FIGS. 8A-8B: Representative data demonstrating that monovalent presentation is sufficient to observe viral neutralization if conjugated to SC. Schematic of assay (FIG. 8A). Dose-response characterization of virus neutralization capacity of SCnbGFP with three different valencies of binders (FIG. 8B). X-axis represents the concentration of snub cube.

FIGS. 9A-9B: Representative data demonstrating that higher valency presentation enhances kinetics of virus neutralization. Schematic of assay (FIG. 9A). All valencies tested saturate at a similar EC50 if incubated with virus for sufficient time (1 hr). Shorter incubation times reveal that higher valencies can exhibit viral neutralization with shorter (or no) pre-incubation with virus (FIG. 9B).

FIGS. 10A-10B: Representative data demonstrating anti-GFP RNA aptamers conjugated to SC exhibit enhanced inhibition of PRV 486. A representative RNA aptamer sequence that binds GFP, was swapped in as a binder in the platform. Both RNA aptamers included a 5′ docking domain, which bind to the respective conjugation sites on the Snub Cube. Two different linker lengths of 6 or 26 base pairs (flex) were used between the docking domain and the aptamer sequence. Aptamer designs conjugated to the snub cube as visualized by a gel shift (FIG. 10A, top). Aptamers bind to PRV486 with similar affinity to EGFP in an ELISA assay (FIG. 10A, bottom). The 4 different aptamer variants were conjugated to snub cube with valencies of 24, 36 and 60 (FIG. 10B). In each instance, conjugation of the aptamer to the snub cube decreased the concentration in which PRV inhibition is observed compared to the aptamer alone.

FIG. 11: Representative data from in vitro cytotoxicity assay. Cell viability after treatment with SC60-nbGFP and individual platform components. Cells were incubated with different concentrations of the antiviral inhibitor or its individual components for 12 h.

FIG. 12: Representative data of serum stability of the multivalent platforms. SC-nbGFP of three different valency: SC4H-nb, SC24H-nb and SC60H-nb were incubated for up to 8 h at 37 C in cell culture medium with increasing concentrations of FBS. The products were analyzed by agarose gel electrophoresis (AGE).

FIGS. 13A-13C: SC-nbGFP blocks viral attachment to host cells. Schematic showing the early entry events of PRV 483 comprising attachment followed by membrane fusion and internalization of virus capsids inside the cytoplasm (FIG. 13A). Attachment of virus particles pretreated with SC60H-nbGFP versus SC60H (control). Data are presented as mean±S.D (FIG. 13B). Representative images from widefield fluorescence microscopy performed on PK15 cells infected with viruses (green) pretreated with either SC60H-nbGFP (top row) or SC60H (bottom row) (FIG. 13C). The figure contains images from the 30 min treatment timepoint. Images from t=0 min and t=60 min can be found in FIG. 14. Cell nuclei are stained with Hoechst (white). The third column represents annotated images from the third column in which the cell area is marked by blue lines and virus particles are marked by green circles.

FIG. 14: SC-nbGFP blocks viral attachment to host cells at various time points. Representative images from widefield fluorescence microscopy performed on PK15 cells infected with viruses pretreated with either SC60H (left) or SC60H-nbGFP (right). The second and fourth columns represent annotated images from the first and third columns. Cell nuclei are stained with Hoechst (white). The cell area is marked with blue outlines. Internalized virus capsids are indicated by red circles.

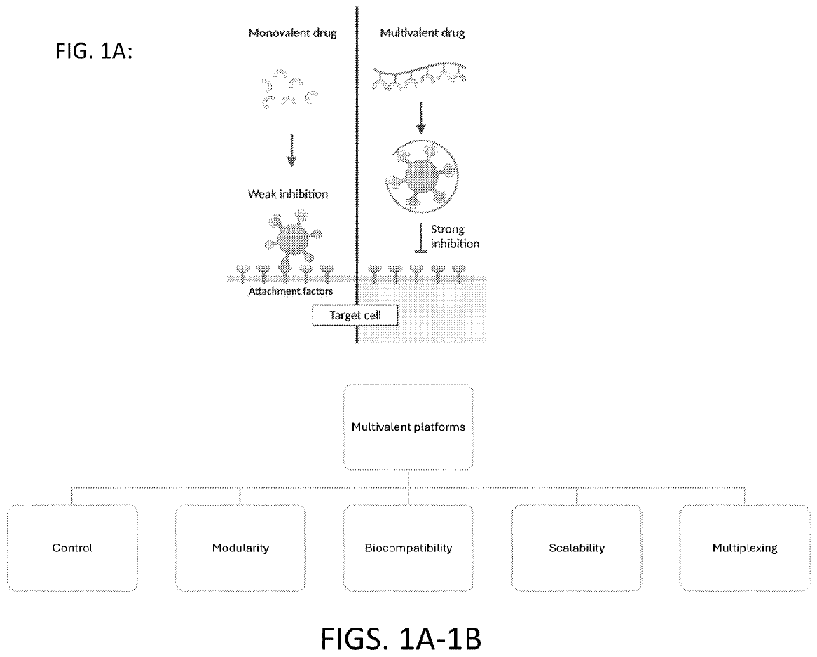

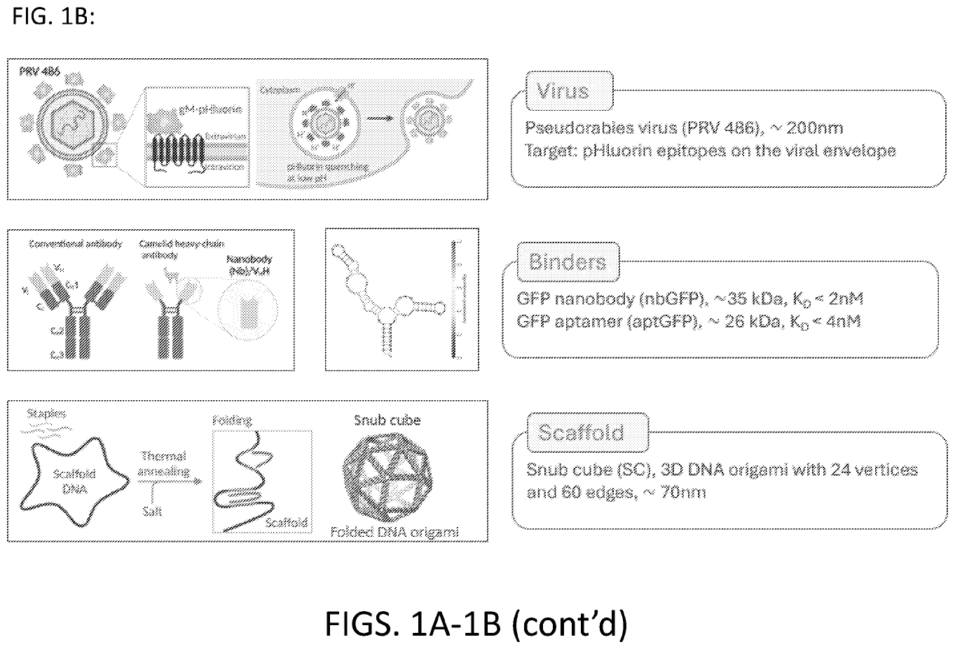

### DETAILED DESCRIPTION

The present disclosure provides compositions and methods related to antiviral therapeutics. In particular, the present disclosure provides a highly modular platform useful for the development and production of multivalent biomolecules capable targeting and neutralizing a virus. In accordance with these embodiments, the present disclosure provides a multivalent antiviral biomolecule comprising a nucleic acid-based scaffold and a plurality of antiviral binders conjugated to the scaffold. In some embodiments, each antiviral binder of the plurality of binder targets one or more viral proteins, thereby neutralizing one or more viruses.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “single-stranded” oligonucleotides generally refers to those oligonucleotides that contain a single covalently linked series of nucleotide residues.

The terms “oligomers” or “oligonucleotides” include RNA or DNA sequences of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. Oligonucleotide” or “oligomer” can also be used to describe artificially synthesized polymers that are similar to RNA and DNA or DNA and RNA molecules with modified backbone and nucleosides, including, but not limited to, oligos of peptide nucleic acids (PNA) and locked nucleic acids (LNA).

The terms “binding activity” and “binding affinity” generally refer to the tendency of a ligand molecule to bind or not to bind to a target. The energetics of these interactions are significant in “binding activity” and “binding affinity” because they can include definitions of the concentrations of interacting partners, the rates at which these partners are capable of associating, and the relative concentrations of bound and free molecules in a solution.

“Sequence identity” refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic (e.g., aspartate, glutamate), basic (e.g., lysine, arginine, histidine), non-polar (e.g., alanine, valine, leucine, isoleucine, proline, phenylalanine, methionine, tryptophan) and uncharged polar (e.g., glycine, asparagine, glutamine, cysteine, serine, threonine, tyrosine). The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

### 2. MULTIVALENT ANTIVIRAL BIOMOLECULES

The present disclosure provides compositions and methods related to antiviral therapeutics. In particular, the present disclosure provides a highly modular platform useful for the development and production of multivalent biomolecules capable targeting and neutralizing a virus. The platform is agnostic to specific virus types, and can be modulated with different antiviral binders to target one or more viruses of interest. In accordance with these embodiments, the present disclosure provides a multivalent antiviral biomolecule comprising a nucleic acid-based scaffold and a plurality of antiviral binders conjugated to the scaffold. In some embodiments, each antiviral binder of the plurality of binder targets one or more viral proteins, thereby neutralizing one or more viruses.

As exemplified further below, the multivalent antiviral platform of the present disclosure includes biomolecules comprising a plurality of antiviral binders conjugated to a nucleic acid-based scaffold that target a candidate virus. For example, a pseudorabies virus (PRV) is the causative agent of Aujeszky's disease which remains an endemic problem among swine populations in many parts of the world. With the rise of vaccine-resistant strains of PRV, control and eradication have been challenging. However, owing to their broad host range and remarkable ability to infect synaptic neurons, they have been widely used as a model organism to study herpesvirus biology and as a transsynaptic tracer. Embodiments of the present disclosure include the development of a recombinant PRV strain 486 that expresses pHluorin (pH-sensitive GFP) on the viral envelope.

Additionally, a nanobody GFP (nbGFP) and GFP aptamers (aptGFP) were developed as virus binders. Nanobodies are heavy chain antibodies that only consist of the variable heavy domain (VHH) and compared to standard antibodies, offer several attractive features like high affinity, low molecular weight, high stability, ease of synthesis, and compatibility with modifications. Aptamers are single-stranded short (<100 nucleotides) DNA or RNA that can fold into unique secondary and tertiary structures with high binding affinity and specificity to their targets. Additionally, because of their low cost, stability, and unlimited target types, they can be very effective as diagnostic and therapeutic tools. Two aptamers were designed: aptGFP1 and aptGFP2, which differ in the length of the spacer between the snub cube and the binder. In the aptGFP2 design, the presence of additional bases between the docking region and the binder imparts flexibility to the binder as opposed to the more rigid double-stranded spacer in the aptGFP1 design.

Additionally, for the scaffold, polymers (polystyrene beads), nanoparticle (quantum dots), and DNA origami-based nanostructures were initially selected. Although approaches using polystyrene beads and quantum dots have certain advantages, embodiments of the present disclosure focused on the use of a nucleic acid-based scaffold, in part because of the ability to control and characterize conjugation. The virus binders were assembled on the snub cube (SC), a 3D wire frame DNA nanostructure that has twenty-four vertices and sixty edges. Single-stranded DNA overhangs extend from each of its sixty edges which can be used for bioconjugation. The copy number of binders can therefore be varied from SC1 to SC60. Here, maximum valency i.e., SC60H, was used initially.

In accordance with the above, multivalent antiviral biomolecules were developed to include a nucleic acid-based scaffold and a plurality of antiviral binders conjugated to the scaffold. In some embodiments, each antiviral binder of the plurality of binder targets one or more viral proteins, thereby neutralizing one or more viruses. In some embodiments, the plurality of antiviral binders target the same viral protein. In other embodiments, the plurality of antiviral binders target two or more distinct viral proteins.

In some embodiments, the nucleic acid-based scaffold comprises a DNA origami-based nanostructure. In some embodiments, the nucleic acid-based scaffold comprises a DNA snub cube. In some embodiments, the DNA snub cube comprises 24 vertices and 60 edges. In some embodiments, at least one edge of the DNA snub cube is conjugated to at least one antiviral binder of the plurality of antiviral binders.

As would be recognized by one of ordinary skill in the art based on the present disclosure, other compositions can be used as a scaffold. For example, the scaffold can be a non-nucleic acid-based scaffold. In some embodiments, the scaffold comprises a polystyrene bead, a quantum dot, and/or a peptide or polypeptide.

As would be recognized by one of ordinary skill in the art based on the present disclosure, any specific number of antiviral binders can be conjugated to the nucleic acid-based scaffold, depending on, for example, the architecture of the scaffold, the virus being targeted, the nature of the antiviral binders being used, among other reasons. In some embodiments, at least about 1 antiviral binders are conjugated to the scaffold. In some embodiments, at least about 2 antiviral binders are conjugated to the scaffold. In some embodiments, at least about 3 antiviral binders are conjugated to the scaffold. In some embodiments, at least about 4 antiviral binders are conjugated to the scaffold. In some embodiments, at least about 8 antiviral binders are conjugated to the scaffold. In some embodiments, at least about 12 antiviral binders are conjugated to the scaffold. In some embodiments, at least about 16 antiviral binders are conjugated to the scaffold. In some embodiments, at least about 20 antiviral binders are conjugated to the scaffold. In some embodiments, at least about 24 antiviral binders are conjugated to the scaffold. In some embodiments, at least about 28 antiviral binders are conjugated to the scaffold. In some embodiments, at least about 32 antiviral binders are conjugated to the scaffold. In some embodiments, at least about 36 antiviral binders are conjugated to the scaffold. In some embodiments, at least about 40 antiviral binders are conjugated to the scaffold. In some embodiments, at least about 44 antiviral binders are conjugated to the scaffold. In some embodiments, at least about 48 antiviral binders are conjugated to the scaffold. In some embodiments, at least about 52 antiviral binders are conjugated to the scaffold. In some embodiments, at least about 56 antiviral binders are conjugated to the scaffold. In some embodiments, about 60 antiviral binders are conjugated to the scaffold.

In some embodiments, the plurality of antiviral binders are conjugated to the nucleic acid-based scaffold via a linker. In some embodiments, the linker comprises a single-stranded DNA overhang coupled to the scaffold. In some embodiments, the linker comprises a single-stranded DNA complementary to the single-stranded DNA overhang. In some embodiments, the complementary single-stranded DNA is conjugated to at least one antiviral binder of the plurality of antiviral binders. In some embodiments, the complementary single-stranded DNA is conjugated to at least one antiviral binder of the plurality of antiviral binders via a SNAP-tag with a benzylguanine (BG) linker.

In some embodiments, at least one of the plurality of antiviral binders comprises a monoclonal antibody, a human antibody, a humanized antibody, a chimeric antibody, and/or a single-domain antibody. In some embodiments, at least one of the plurality of antiviral binders comprises an antibody fragment selected from the group consisting of Fab, Fab-C, Fab′-SH, Fv, scFv, and (Fab′)₂ fragments. In some embodiments, at least one of the plurality of antiviral binders comprises a single-domain antibody. For example, the Camelid species possess unusual heavy chain IgG antibodies, devoid of light-chains and containing a single antigen binding variable domain (VHH). This VHH domain is also referred to as a nanobody. A nanobody is strictly monomeric, highly stable, and generally smaller than the VH variable domain of a classical antibody and can be readily expressed in heterologous systems such as bacteria at high levels.

In some embodiments, at least one of the plurality of antiviral binders comprises an aptamer. In some embodiments, the aptamer is an RNA or DNA aptamer. In some embodiments, the aptamer comprises a docking domain to facilitate conjugation to the scaffold. In some embodiments, the aptamer further comprises a linker region between the docking domain and a region of the aptamer that binds a viral protein. In some embodiments, the linker region is from about 2 to about 40 base pairs in length. In some embodiments, the linker region is from about 4 to about 40 base pairs in length. In some embodiments, the linker region is from about 6 to about 40 base pairs in length. In some embodiments, the linker region is from about 8 to about 40 base pairs in length. In some embodiments, the linker region is from about 10 to about 40 base pairs in length. In some embodiments, the linker region is from about 15 to about 40 base pairs in length. In some embodiments, the linker region is from about 20 to about 40 base pairs in length. In some embodiments, the linker region is from about 25 to about 40 base pairs in length. In some embodiments, the linker region is from about 30 to about 40 base pairs in length. In some embodiments, the linker region is from about 35 to about 40 base pairs in length. In some embodiments, the linker region is from about 2 to about 35 base pairs in length. In some embodiments, the linker region is from about 2 to about 30 base pairs in length. In some embodiments, the linker region is from about 2 to about 25 base pairs in length. In some embodiments, the linker region is from about 2 to about 20 base pairs in length. In some embodiments, the linker region is from about 2 to about 15 base pairs in length. In some embodiments, the linker region is from about 2 to about 10 base pairs in length. In some embodiments, the linker region is from about 10 to about 30 base pairs in length. In some embodiments, the linker region is from about 10 to about 20 base pairs in length. In some embodiments, the linker region is from about 20 to about 30 base pairs in length.

Embodiments of the present disclosure also include a composition comprising a plurality of any of the antiviral biomolecules described herein. In some embodiments, the composition includes a pharmaceutically acceptable adjuvant, excipient, and/or carrier, and wherein the composition is suitable for administration to a subject in need thereof. For example, the subject can be in need of therapeutic antiviral treatment, and/or the subject can be in need of prophylactic antiviral treatment.

Embodiments of the present disclosure also include a method of detecting a target virus using any of the antiviral biomolecules described herein. For example, the antiviral biomolecules of the present disclosure can be used in an in vitro assay to detect the presence of a target virus, including but not limited to, virus neutralization assays. In accordance with this embodiments of the present disclosure also include a method of neutralizing a target virus using any of the antiviral biomolecules described herein. Embodiments of the present disclosure also include a kit comprising any of the biomolecules described herein, and instructions for detecting or neutralizing a target virus.

### 3. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Despite their transformative use in healthcare, antivirals have been clinically approved to treat only 10 out of the more than 200 known pathogenic human viruses. Additionally, many virus functions are intimately coupled with host cellular processes, which present challenges in antiviral development due to the limited number of clear targets per virus, necessitating an extensive insight into these molecular processes. Compounding this challenge, many viral pathogens have evolved to evade effective antivirals. Accordingly, there is a need for effective antivirals for a variety of human viruses.

Provided herein is a multivalent antiviral molecule composed of a viral binder and a bulky scaffold that sterically blocks interactions between a viral particle and a host cell. The multivalent antiviral molecule platform used herein is suitable for the development of antivirals agnostic to the extravirion epitope that is being bound. Specifically, a nanobody that specifically recognizes a non-essential epitope presented on the extra virion surface of Pseudorabies virus strain 486 was modified with a 3-dimensional wireframe DNA origami structure ~100 nm in diameter. The nanobody switches from having no inhibitory properties (tested up to 50 M) to ~3 nM IC50 when conjugated with the DNA origami scaffold. Mechanistic studies support that inhibition is mediated by the non-covalent attachment of the DNA origami scaffold to the virus particle, which obstructs the attachment of the viruses onto host cells.

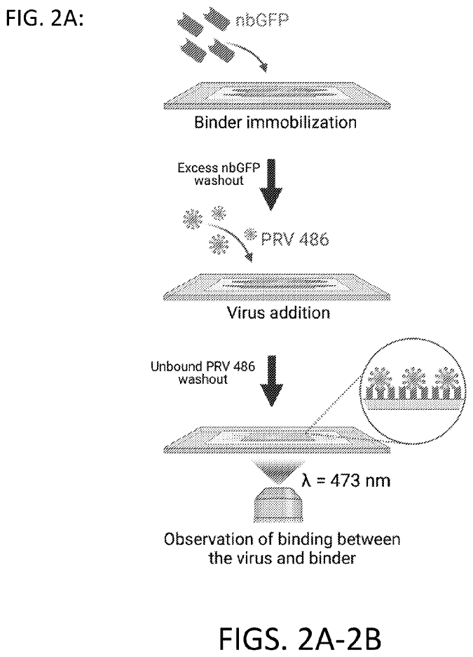

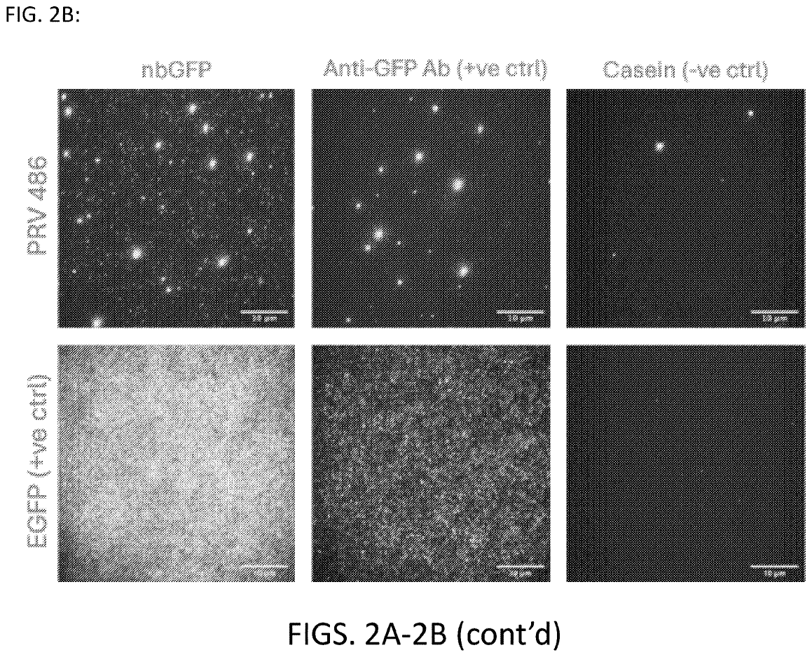

Choice of virus binders to target PRV486. Flow cells were made on plasma-cleaned Corning D263 glass slides (24 mm×50 mm) using a double-sided Kapton tape and coated them with nbGFP. After blocking with 0.1% casein, PRV 486 was added to the flow cells. Next, flow cells were sealed with a clean Biotium coverslip (20 mm×20 mm) and imaged using an Oxford Nanoimager microscope (ONI) with a 473 laser at low power (2% or <20 mW), TIRF angle 55°, and exposure of 100 ms. Flow cells without nbGFP were used as a negative control for nbGFP, anti-GFP antibody (AbGFP) as a positive control for nbGFP, and EGFP protein as a positive control for PRV 486. In the presence of binding, PRV 486 immobilizes on the flow cell which can be visualized under the microscope using their pHluorin tags (FIGS. 2A-2B).

The immobilization of PRV 486 on flow cells coated with nbGFP is clearly indicated by the distinct white dots against the black background on the fluorescent images. The white dots are also present on flow cells coated with AbGFP (positive control for nbGFP). The lack of white dots reflected in the negative control group shows negligible virus immobilization in the absence of nbGFP (FIGS. 2A-2B).

### Design, Synthesis, and Characterization of DNA Origami-Based Multivalent Binder

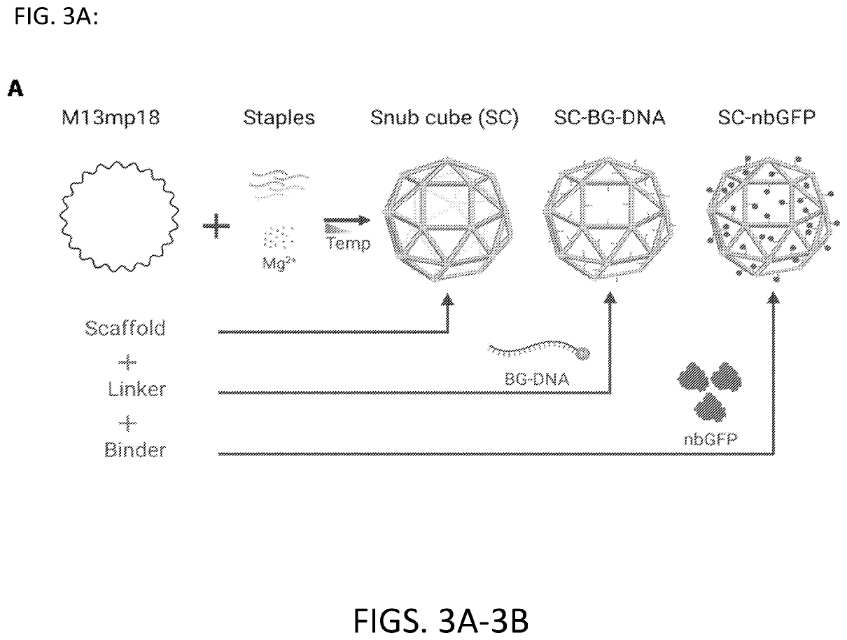

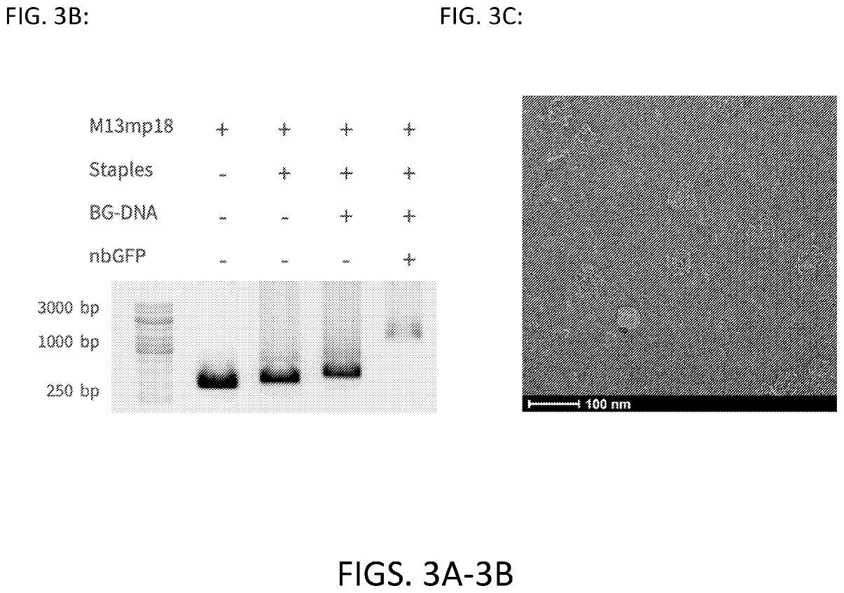

A 3D DNA origami scaffold was chosen to control the spatial distribution of virus binders with defined nanometer-level precision and to minimize batch-to-batch variations of the assembled nanostructures (FIG. 3A). Increasing the size of the DNA origami scaffold to match the size of the virus may increase the antiviral efficacy of the multivalent system and provide a higher surface area to increase the valency without clustering of ligands. However, a larger DNA origami nanostructure may compromise its immunotolerance and necessitate a higher Mg2+ ion concentration to maintain its structural integrity, which is not typical in physiological environments. To rationally design the 3D scaffold, considering the size range of virus particles, maintaining the structural integrity of the assembled multivalent system, and maintaining a low surface area to volume ratio, a wireframe DNA origami scaffolds, 50-100 nm diameter size, was used.

The snub cube (SC), a 3D wireframe DNA origami, 60 nm diameter size, with 60 edges, 24 vertices, and 38 faces, including 6 squares and 32 equilateral triangles, was used. SC is built using a 7.25 kb long strand (M13mp18) hybridized to 192 staple oligonucleotides. It was self-assembled using a one-pot thermal annealing reaction and purified using molecular weight cut-off (MWCO 100 kDa) filtration. 20-nt single-stranded DNA (ssDNA) overhangs in the middle of its edges were incorporated into the SC. To functionalize SC with virus binders, one or more copies of the SNAP-tag nbGFP per SC (one binder on one edge of the SC) were site-specifically conjugated via benzyl guanine (BG) linkers (FIG. 3C). Using this approach, the copy number of virus binders in the SC scaffold could be varied from 1 to 60, assuming a 1:1 stoichiometric ratio of binding between the nbGFP and SC edges. To span the range between 1 and 60, SC-nbGFP was constructed of three valences, including SC1H-nbGFP, SC12H-nbGFP, and SC60H-nbGFP. A 12.5 mM Mg²⁺ concentration was maintained throughout all preparation and purification steps to retain the structural integrity of the assembled nanostructures. The formation of SC, SC-BG-DNA, and SC-nbGFP was characterized after each step using agarose gel electrophoresis. The formation of distinct bands and the reduced electrophoretic mobility of these bands were consistent with their relatively increasing molecular weights, confirming the correct formation of nanostructures after each synthesis step. The final SC-nbGFP exhibited the lowest mobility in gel electrophoresis, followed by SC-BG-DNA intermediate nanostructures with the complementary ssDNA attached to the BG linker, and finally by the SC constructs with 60 ssDNA overhangs corresponding to their valency. In addition, SC60H-nbGFP was characterized using dynamic light scattering (DLS) and found a majority peak at 100 nm in diameter.

Multivalent Conjugation of nbGFP to SC Enhances Virus Binding

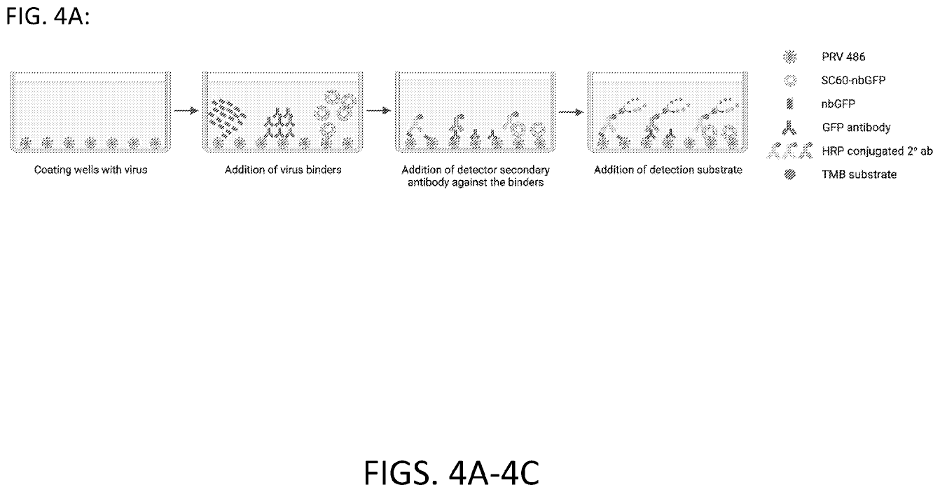

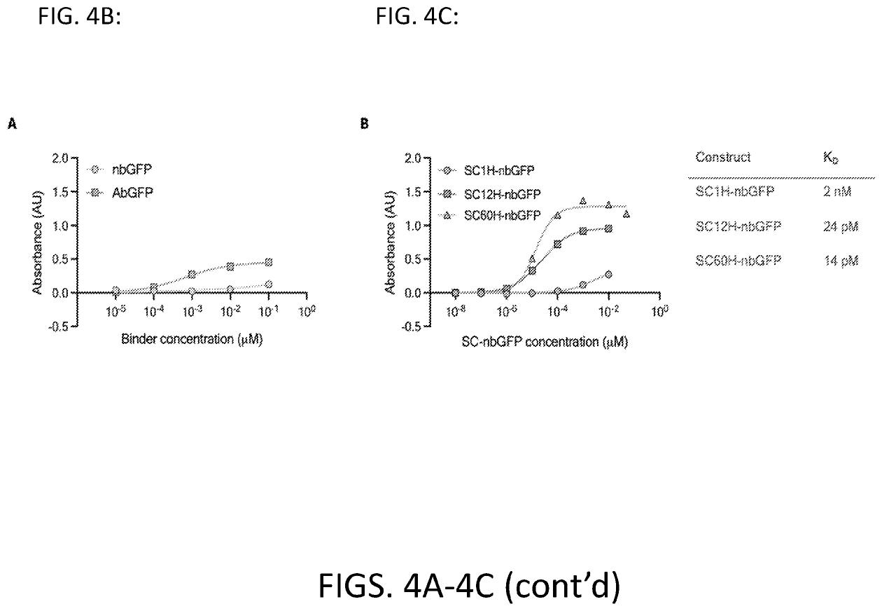

To address the functionality of the multivalent system, the impact of SC-nbGFP valency on virus binding was evaluated using semiquantitative ELISA assays (FIG. 4A). 96-well ELISA plates were coated with PRV 486 and incubated them with monomeric nbGFP and multivalent SC-nbGFP nanostructures of different valences. The extent of binding of nbGFP and SCnbGFP nanostructures using an orthogonal nbGFP-specific reporter antibody.

The monomeric nbGFP, monovalent SC1H-nbGFP, and multivalent SC12H-nbGFP and SC60H-nbGFP showed concentration-dependent changes in signal intensities, suggesting specific binding activity (FIG. 4B). nbGFP exhibited a relatively low affinity for the pHluorin moiety on PRV 486, possibly due to the sequence differences between pHluorin and EGFP. The low binding of nbGFP at concentrations <10 nM may also be attributed to inefficient passivation of the virus surfaces to prevent nonspecific interactions (FIG. 4B). The multivalent interactions of SC12H-nbGFP and SC60H-nbGFP increased the overall binding affinity. The non-linear fits are the specific binding with Hill slope h given by

$$Absorbance = {B}_{ma x} ( \frac{{[ binder ]}^{h}}{{[ binder ]}^{h} + {K}_{D}^{h}} ) ,$$

where h is the Hill slope, Bₘₐₓ is the effective maximum specific binding, and K_{D} is the binder concentration needed to achieve a half-maximum binding at equilibrium. The relative K_{D}'s showed a 90-fold increase in the binding affinity of SC12H-nbGFP (24±1 μM) and a 150-fold increase in the binding affinity of SC60H-nbGFP (14±4 μM) compared to that of monovalent SC1H-nbGFP (2200±200 μM; FIG. 4C) and GFP antibody (670±10 μM; FIG. 4B). These data indicate that nbGFP targets the pHluorin-tagged gM domains of the PRV 486, and multivalent conjugation of nbGFP onto SC enhanced the binding strength of the SC-nbGFP complex with PRV 486.

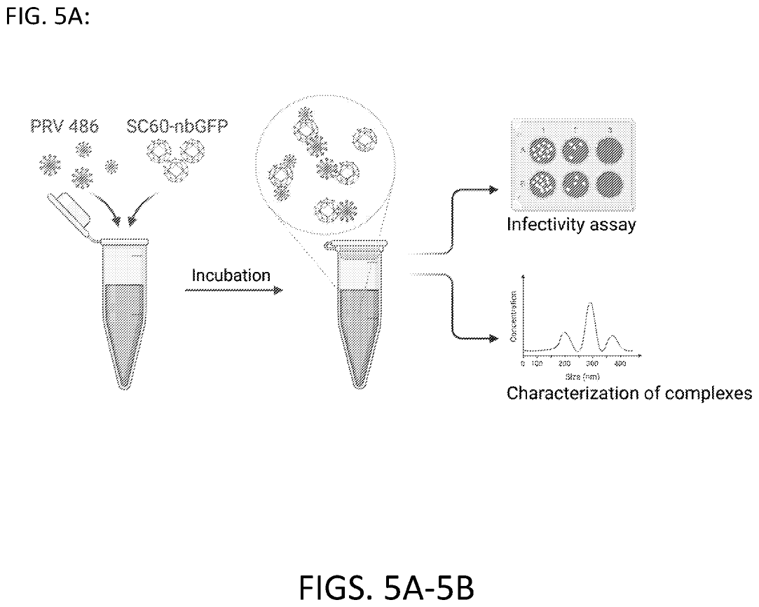

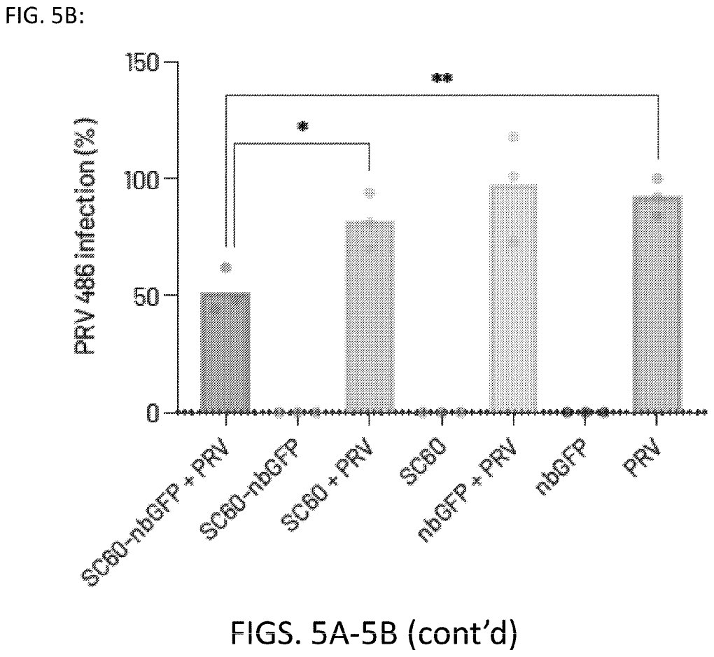

Presentation of nbGFP on SC enables viral neutralization. Next, it was investigated whether the multivalent platform could influence viral infectivity in vitro. PRV 486 and SC60-nbGFP were incubated at 1:1000 molar ratio and performed plaque-neutralization assays. The assay was done using the pig kidney cell line (PK15) following standard protocol. The results are shown in FIGS. 5A-5B. The infective of PRV only group was used as reference to calculate residual infectivity of all other groups. As can be seen, at 1000× molar excess of the SC60-nbGFP, the viral infectivity was reduced by 51% in comparison to the no inhibitor (PRV only) group. Scaffold only (SC60) and binder only (nbGFP) were employed as negative controls, each of which showed negligible effect on viral infectivity.

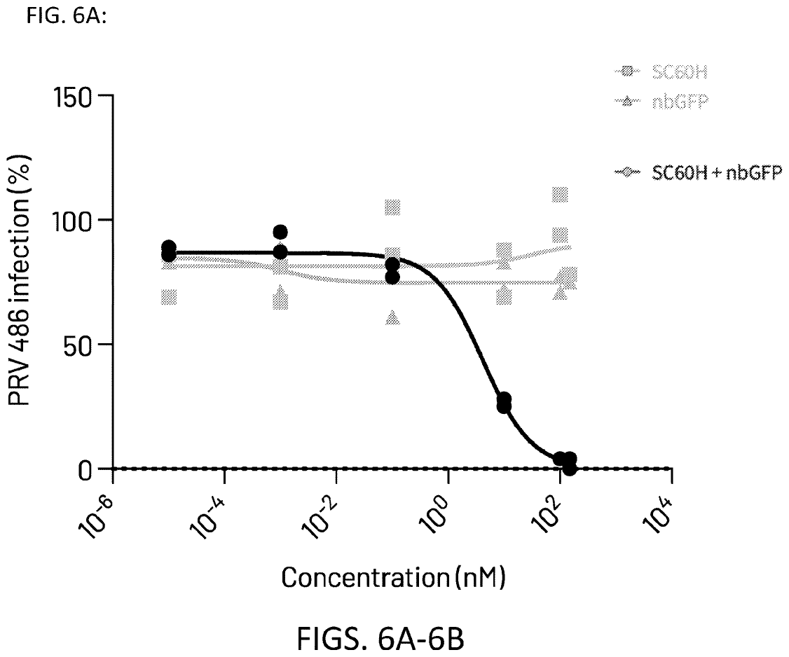

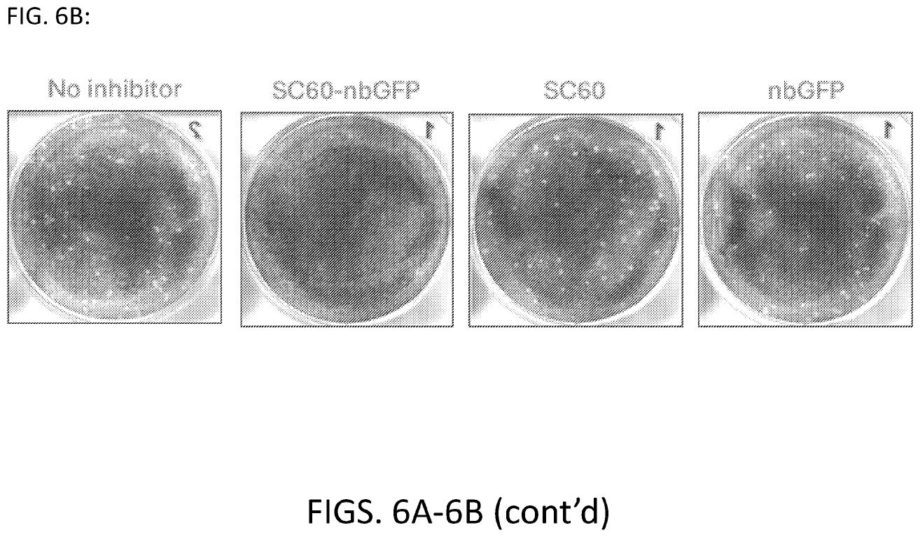

nbGFP only exhibits viral neutralization when conjugated to SC. Next, PRV neutralization capacity of SC60-nbGFP was investigated in vitro through standard antiviral, plaque-forming half-maximum inhibitory concentration (IC₅₀) assays. In brief, PRV was incubated with different concentrations of SC60-nbGFP in cell culture media and determined the remaining infectivity by plaque reduction assay. The inhibition of PRV by SC60 and nbGFP was examined as controls. The results are summarized in FIGS. 6A-6B. As can be seen. SC60-nbGFP demonstrated dose-dependent inhibition of PRV with IC₅₀ value of 4 nM (N=2). In comparison, SC and nbGFP showed no inhibition. This experiment demonstrates the ability of the platform to leverage multivalency to offset weak interactions and subsequently neutralize viral infection.

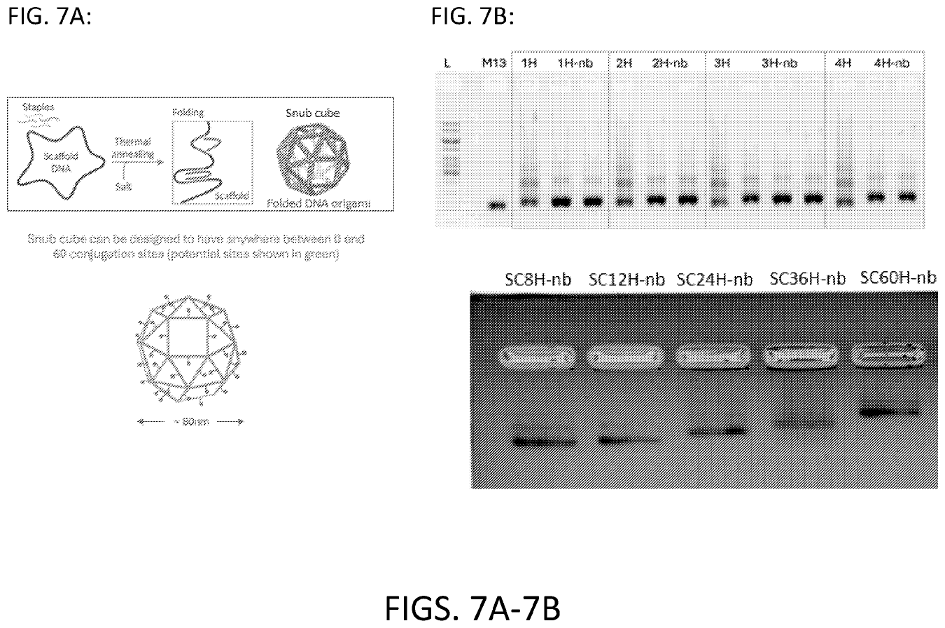

The valency of nbGFP on the SC can be controlled. Representative data using standard design principles of DNA origami in which the number of ssDNA conjugation sites can be designed (FIG. 7A). Conjugation of varying amounts of nanobody (nb) per snub cube is evaluated in gel shift assay (FIG. 7B). Four valencies are evaluated, 1, 2, 3 and 4. For each, the snub cube with corresponding valency along (nH) is run next to two repeats when conjugated nanobody. Conjugated snub cubes with valencies of 8, 12, 24, 36 and 60 are run next to each other. Gel shifts are observed to generally correlate with the degree of valency (FIG. 7B).

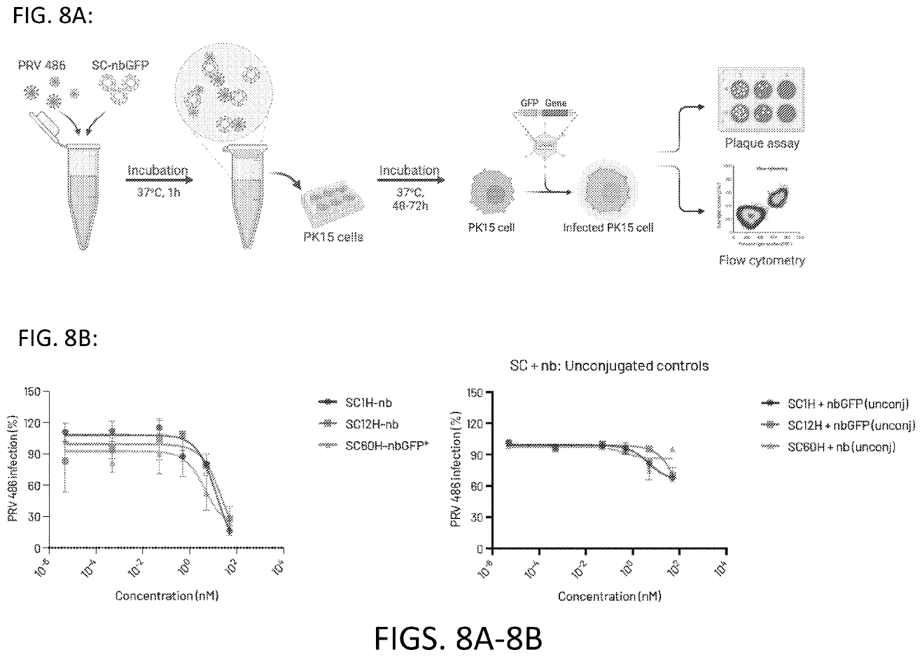

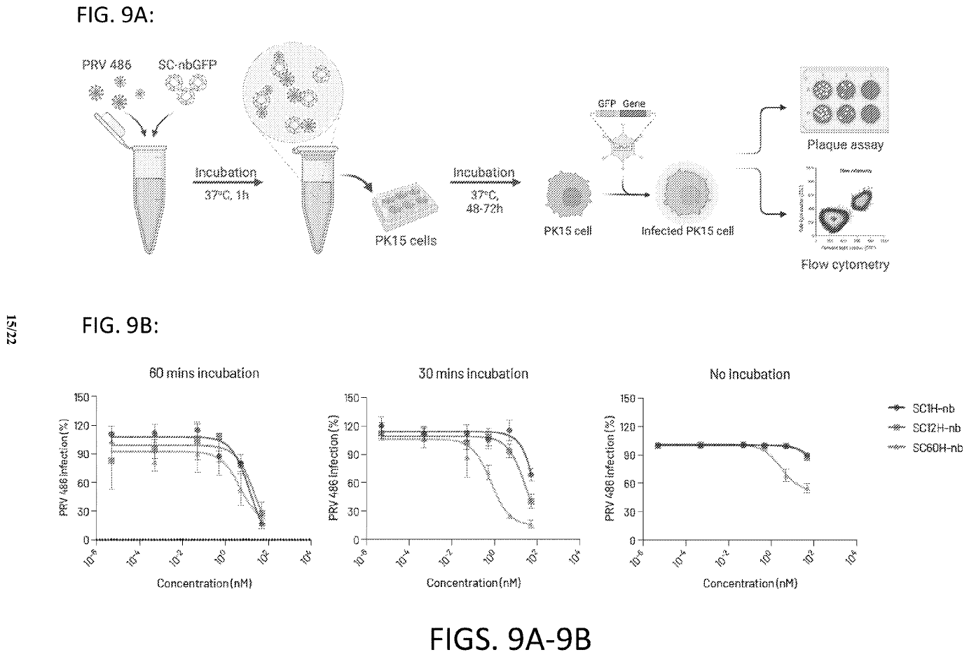

Experiments were also conducted to demonstrate that monovalent presentation is sufficient to observe viral neutralization if conjugated to SC. Schematic of assay (FIG. 8A). Dose-response characterization of virus neutralization capacity of SCnbGFP with three different valencies of binders (FIG. 8B). X-axis represents the concentration of snub cube. Additionally, as shown in FIGS. 9A-9B, data demonstrated that higher valency presentation enhances kinetics of virus neutralization. All valencies tested saturate at a similar EC50 if incubated with virus for sufficient time (1 hr). Shorter incubation times reveal that higher valencies can exhibit viral neutralization with shorter (or no) pre-incubation with virus (FIG. 9B).

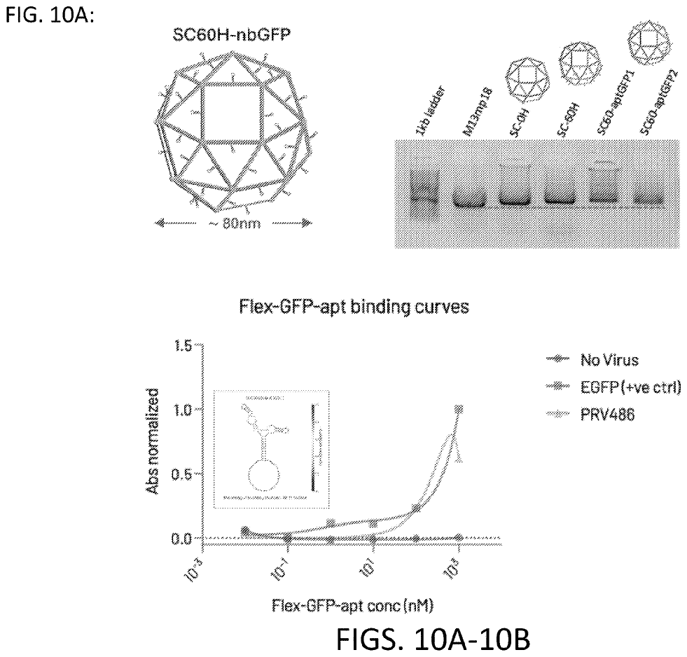

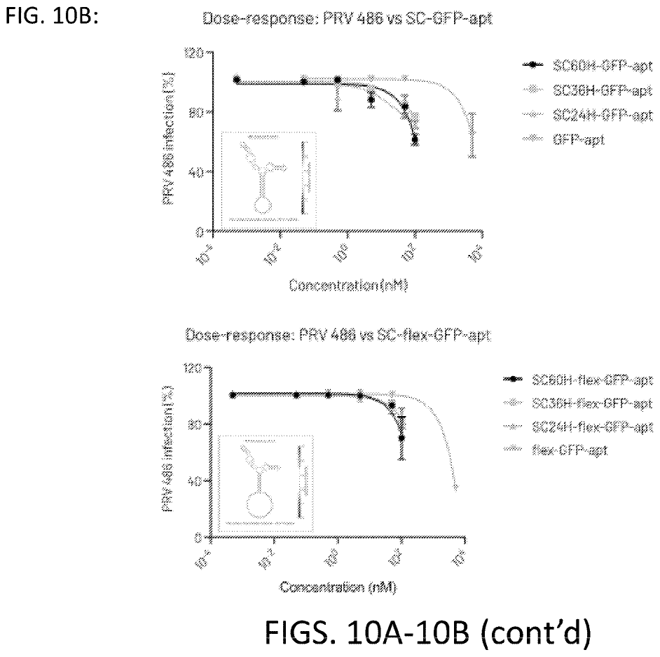

Anti-GFP RNA aptamers conjugated to SC exhibit enhanced inhibition of PRV 486. Experiments were also conducted to demonstrate that anti-GFP RNA aptamers conjugated to SC exhibit enhanced inhibition of PRV 486. Two different RNA aptamer sequences, both of which bind GFP were swapped in as binders in the platform. Both RNA aptamers included a 5′ docking domain, which bind to the respective conjugation sites on the Snub Cube. Two different linker lengths of 6 or 26 base pairs (flex) were used between the docking domain and the aptamer sequence. Aptamer designs conjugated to the snub cube as visualized by a gel shift (FIG. 10A, top). Aptamer binds to PRV486 with similar affinity to EGFP in an ELISA assay (FIG. 10A, bottom). The 4 different aptamer variants were conjugated to snub cube with valencies of 24, 36 and 60 (FIG. 10B). In each instance, conjugation of the aptamer to the snub cube decreased the concentration in which PRV inhibition is observed compared to the aptamer alone.

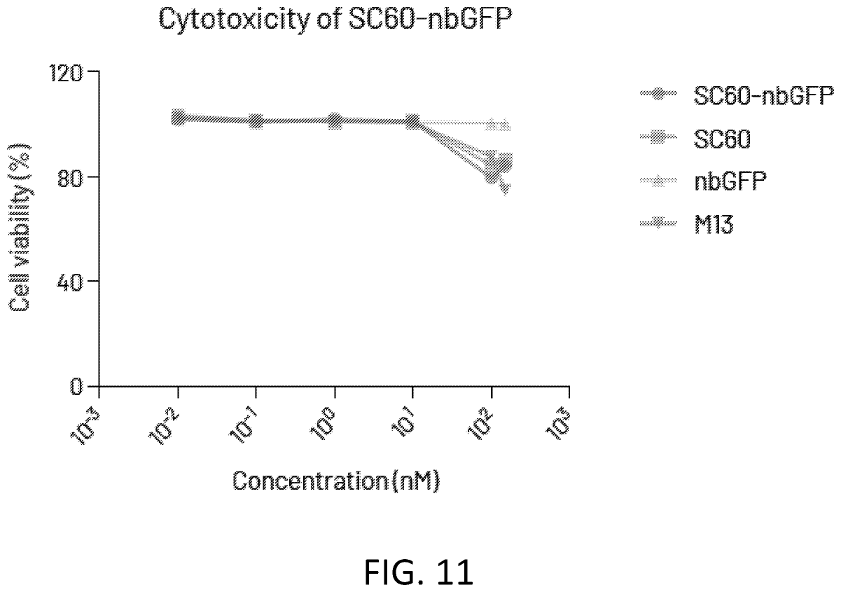

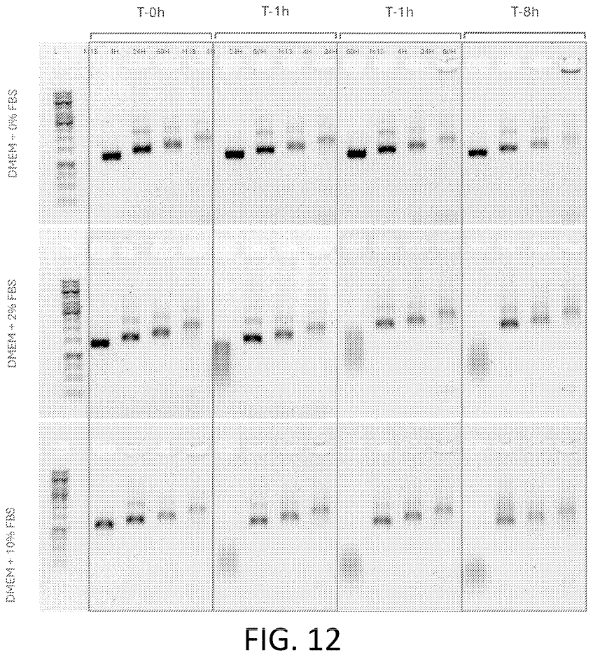

Minimal Cytotoxicity of SC60H-nbGFP is observed at inhibitory concentrations. To further substantiate the therapeutic efficacy of the antiviral platform, a cytotoxicity assay was performed. The PK15 cell line was examined by LDH assay to assess the effects of cytotoxicity of SC60-nbGFP and individual platform components: SC60 and nbGFP. The results are summarized in FIG. 11. As can be seen, for the range of concentrations that were tested, nbGFP exhibited no obvious cytotoxicity towards the cells; almost 100% of the cells were still viable after 24 h incubation with nbGFP. SC and SC60-nbGFP on the other hand were well tolerated at concentrations <10 nM. With increasing concentrations of SC60-nbGFP and SC, the cell viability dropped down to ~85%, and further down to ~75%, when the concentration reached 150 nM.

### SC-nbGFP Obstructs Viral Attachment to Host Cell Surfaces

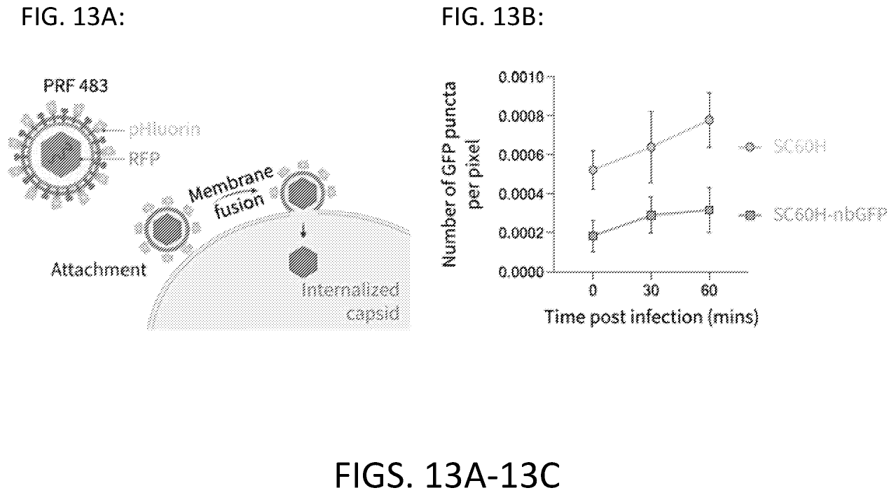

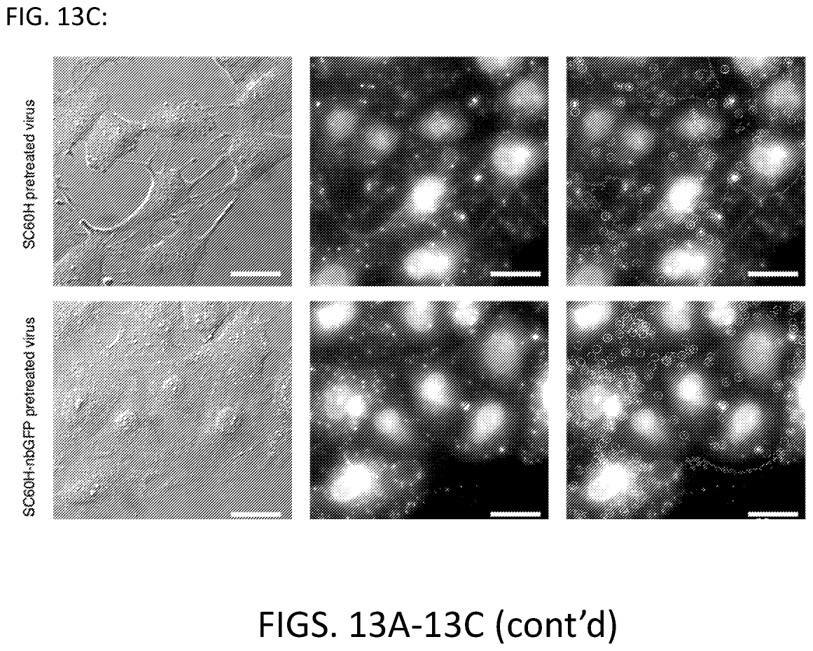

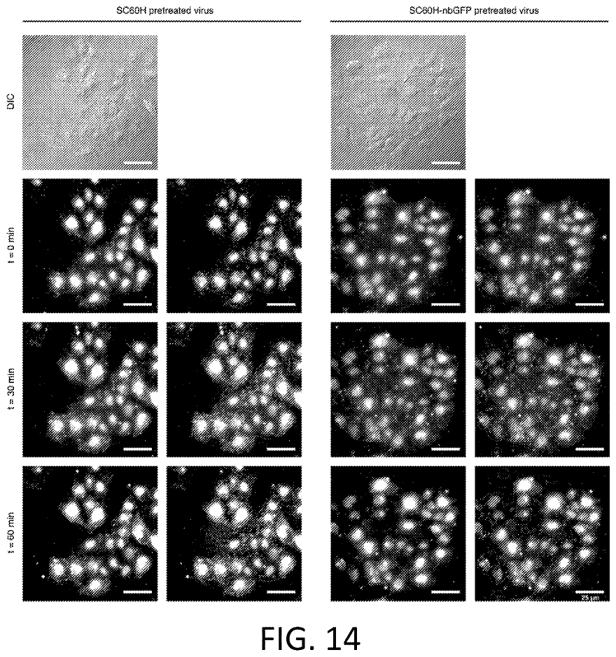

It was next assessed whether multivalent interactions would obstruct the entry events of PRV particles, resulting in reduced attachment, reduced internalization, or both. To assess whether SC-nbGFP blocks PRV 486 surface attachment to host cells, time-lapse live-cell fluorescence imaging was performed to track the early events of virus infection. To this end, PRV 483, a different recombinant strain expressing gM-pHluorin on the virus envelope, and a red fluorescent mRFP-VP26 virus capsid tag, were used. At neutral pH, PRV 483 particles exhibit colocalized green and red fluorescence in the extracellular space but shed their virion envelope and exhibit red-only fluorescence after internalization (FIG. 13A). Using PRV 483, colocalized green and red extracellular puncta versus internalized red-only puncta were monitored at 0-, 30-, and 60-mins post-infection. To be able to see a sufficient number of virus particles in this experiment, a multiplicity of infection (MOI) of 104 infectious units per cell was used. To quantify attachment efficiency, the total GFP puncta per unit area was counted, comparing two experimental conditions: PRV 483 pre-incubated with SC-nbGFP, and PRV 483 pre-incubated with SC alone. When treated with SC-nbGFP, significantly fewer green puncta (pHluorin) per unit area were observed compared to the control condition. The untreated viruses showed a 2- to 3-fold greater accumulation of pHluorin puncta per unit area over time (FIG. 13B). However, SC-nbGFP did not decrease the number of internalization events per attached particle compared to the control (FIG. 14). These data indicate SC-nbGFP obstructs the attachment of virus particles onto the host cell. Based on this observation, the moiety was termed a “Viral Attachment Blocking Chimera” (“VirABloC”).

### Example 2

### Exemplary Methods

### Chemicals and Kits

Tris-acetate EDTA (TAE) buffer, magnesium chloride hexahydrate (MgCl₆·H₂O), methylene blue, pluronic-F127, casein, dimethyl formamide (DMF), agarose and polyethylene glycol 8000 (PEG8000), triethylamnmoniurn acetate (TEAA) were purchased from Sigma Aldrich. Cell culture consumables were purchased from Corning. μ-slide 8-well plates for confocal imaging were purchased from Ibidi. DNA ladders, SYBR gold dye, anti-His HRP, streptavidin-HRP, and TMB substrate were purchased from Thermo Fisher. All ELISA reagents (except for TMB) were purchased from Bethyl Laboratories. The BG-GLA-NHS (#S9151S) was purchased from New England Biolabs. The CytoTox 96 non-radioactive cytotoxicity assay kit (#G1780) was purchased from Promega.

### Oligonucleotides and DNA Templates

A total of 16 DNA origami nanostructures (9 conjugated with nbGFP, 6 conjugated with GFP aptamers, and 1 without handles) were designed using Tiamat. All DNA staple strands used for assembling scaffolded snub cube (SC) DNA origami nanostructures and modified DNA oligonucleotides for docking virus binders onto the SC scaffold were purchased in 96-well plates from Integrated DNA Technologies at 100 nmol synthesis scale with concentrations normalized to 500 μM. The M13mp18 single-stranded DNA scaffold was produced in-house using.

### Cell Culture and Virus Propagation

PK15 cells, a transformed porcine kidney cell line, were obtained from ATCC (#CCI-33). Cells were maintained in complete Dulbecco's modified Eagle's medium (DMEM) supplemented with 10% fetal bovine serum (FBS), 4 mM L-glutamine, 100 U/mL penicillin, and 100 μg/mL streptomycin in a humidified 37° C., 5% CO₂ incubator.

Viruses were propagated in PK15 cells as follows: PK15 cells were grown in complete medium to 90% confluency in a sterile 10 cm cell culture dish. After removing the media and washing the cells once with phosphate-buffered saline, they were infected with PRV at a multiplicity of infection (MOT) of 0.01 infectious units per cell, in a final volume of 1 mL. The cells were incubated to allow virus adsorption for 1 h in a humidified 37° C., 5% CO₂ incubator. After 1 h, the infection inoculum was removed and replaced with 10 mL of fresh virus medium (DMEM supplemented with 2% FBS, 4 mM L-glutamine, 100 U/mL penicillin, and 100 μg/mL streptomycin). The cells were incubated further until 80-90% cytopathic effects were observed, at which point the cells and supernatant were harvested. The mixture was centrifuged at 2000×g for 5 min to remove the cell debris, and the virus supernatant was divided into 100-200 mL aliquots and stored at −80° C. Virus stocks stored at −80° C. were thawed in a 37° C. water bath and sonicated in a cup sonicator (10 pulses, one second on and one second off for a total of 20 seconds at an amplitude of 80%) prior to use.

### Plaque Assay

PK15 cells were seeded in 6-well plates at a density of 4×10 cells per well. The next day, cells were washed once with phosphate-buffered saline and infected with 10-fold serial dilutions of virus samples. Cultures were incubated for 1 h in a humidified 37° C., 5% CO₂ incubator to allow virus adsorption. The unbound virus was removed and replaced with 3 mL of methocel overlay medium (virus medium supplemented with 2% hydroxypropylmethylcellulose). At 3 days post-infection, the methocel overlay medium was removed and cultures were fixed and stained with a 70% methanol solution containing methylene blue dye and incubated at room temperature for up to 24 hours. The staining solution was removed, rinsed with water, and air-dried. To determine the infectious virus titer, the total plaque count was divided by the total volume plated, based on the lowest dilution giving a countable number of plaques, and multiplied by the reciprocal of the corresponding dilution factor.

For the plaque reduction assay, approximately 100-200 infectious units of PRV 486 were mixed with different concentrations of SCnbGFP in a final volume of 200 μL DMEM (without supplements) and incubated at 37° C. for 1 hour. A plaque assay was then performed as described above. The residual infectivity (%) was calculated using the control condition as the reference. Inhibition data was fit by nonlinear regression to determine the half-maximal inhibitory concentration dosage (IC₅₀).

TIRF Fluorescence Microscopy-Based Detection of Interactions Between Viruses and nbGFP

Coming glass coverslips were used to make flow cells for this experiment. The coverslips were cleaned by sonicating in ethanol, rinsing in ultrapure deionized water, sonicating in acetone, incubating in ethanol, rinsing in ultrapure deionized water, incubating in 2% Hellmanex III cleaning solution, rinsing in ultrapure deionized water, and drying under filtered nitrogen gas.

Coverslips were then plasma cleaned for 10 minutes (Harrick Plasma; PDC-32G). Immediately after, flow cells were assembled by sandwiching double-sided Kapton tape between a larger and a smaller coverslip. The Kapton tape was cut to include two channels for replicate testing.

10 μL of 5 μM nbGFP was injected into the flow cells and incubated for 10 minutes in a humidity chamber. All subsequent wash steps were performed using 200 μL of phosphate-buffered saline. After washing excess nbGFP, 1 mg/mL. casein was injected into the flow cells and incubated for 10 minutes to block nonspecific binding. Excess casein was washed out, and 10 μL of 100 μM PRV 486 was added to the flow cells. As a positive control, 100 nM EGFP was used. The samples were incubated for 10 minutes, and unbound material was washed out. The flow cells were sealed with a coverslip sealant and incubated in a humidity chamber before and in between imaging. Flow chambers were then imaged using an Oxford Nanoimager microscope (ONI) with a 473 nm laser at 2% intensity, or <20 mW, a TIRF angle of 55°, and an exposure of 100 ms.

### Assembly of Snub Cube-Nanobody GFP Nanostructures (SC-nbGFP)

The DNA SC used for conjugation to nbGFP were self-assembled in a one-pot reaction in which a 100 nM M13 scaffold was mixed with a 10-fold molar excess of common staple strands, a 10-fold molar excess of the handles corresponding to the valency of the SC, a 10-fold molar excess of the handles which block the remaining spots corresponding to the valency of the SC, and 1 mM TAE+12.5 mM MgCl₂. A final reaction volume of 100 μL was annealed in a thermocycler with the following program: 95° C. for 5 mins; 90° C. to 86° C. at a rate of 4° C. per 5 minutes; 85° C. to 70° C. at a rate of 1° C. per 5 minutes; 70° C. to 40° C. at a rate of 1° C. per 15 minutes; 40° C. to 25° C. at 1° C. per 10 minutes; and hold at 4° C. at the end of the cycle.

Following annealing, the SC nanostructures were purified from excess staple strands using 100 kDa Amicon spin-column filtration. Columns were passivated for 2 minutes with 500 μL of 10% Pluronic-F127 in 1×TAE+12.5 mM MgCl₂ and centrifuged at 16000×g for 10 minutes. The columns were washed with 500 μL of 1×TAE+12.5 mM MgCl₂ before adding the samples and an additional 1×TAE+12.5 mM MgCl₂ up to 500 μL. The columns were spun at 1000×g for 15 minutes before replenishing the 1×TAE+12.5 mM MgCl₂ to 500 μL.

The purified SC scaffolds were mixed with a 10-fold molar excess of BG-conjugated complementary DNA strands and incubated for 90 mins at 37° C. Following annealing, another 2 rounds of 100 kDa Amicon spin-column filtration with passivation were performed following the same procedure described above. Finally, the SC-BG-DNA was mixed with nbGFP at a 5× molar excess of the SC valency in a solution of 1×PBS+12.5 mM MgCl₂+1 mM DTT and incubated overnight at 4° C. with gentle rotation. A final set of 5 rounds of 100 kDa Amicon spin-column filtration with passivation was performed with 1×PBS+12.5 mM MgCh used as the wash buffer in place of 1×TAE+12.5 mM MgCl₂ and all steps were performed at 4° C. to preserve the stability of the assembled SC-nbGFP. Gel electrophoresis was performed following each purification step to confirm the assembly of the DNA origami nanostructures.

### Agarose Gel Electrophoresis

DNA nanostructures were analyzed by agarose gel electrophoresis to assure purity and confirm conjugation. Samples were loaded into a 1% agarose gel according to the following mixture: 1 μL sample, 3 μL ultrapureH₂O, 1 μL 6× loading dye, and 1 μL 6×SYBR GOLD dye. Along with a 1 kb plus DNA ladder, the samples were run in a buffer of 1×TAE+12.5 mM MgCl₂ for 1 hour at 100 V. Gels were imaged with a Bio-Rad Molecular Imager Gel Doc XR System transilluminator at the SYBR GOLD excitation wavelength (495 nm).

### Synthesis of Benzylguanine Conjugated DNA Oligonucleotides

3′-amine modified (3AmMO) single-stranded DNA oligonucleotides complementary to the SC overhang handles were dilated in 0.1 M HEPES pH 8.5 to a final concentration of 1 mM. N-hydroxysuccinimide ester-functionalized benzyl guanine (BG-GLA-NHS) was freshly reconstituted in DMF to a 50 mM final concentration. For conjugation, the two solutions were mixed in an oligonucleotide-amine: BG-GLA-NH4S molar ratio of 1:10. The final concentration of HEPES was maintained between 50 mM and 100 mM. The reaction was incubated at 4° C. for 16 hours with continuous rotation. After incubation, the reaction was desalted using Bio-Rad micro spin columns and further purified using reverse-phase HPLC to remove unconjugated DNA amine. 100 mM TEAA and 100% methanol were used as the HPLC buffers. The HPLC-purified fractions were lyophilized and reconstituted in water. The concentration of BG-conjugated DNA was determined using a NanoDrop spectrophotometer.

### Purification of BG-Conjugated DNA Using Reverse Phase HPLC

BG-conjugated DNA strands were purified from unreacted DNA using a C-18 column on an Agilent 1220 Infinity LC-HPLC system. Sample DNA mixtures were injected into the column in 50-100 ml volume. The purification was performed using a linear gradient method, with Buffer-A (100 mM TEAA) and Buffer-B (100% methanol). The gradient was run from 10% to 100% of Buffer-B over 40 minutes. Migration of the DNA and DNA conjugates were monitored using absorbance at 260 nm. Purified volumes of DNA conjugates were collected and further confirmed for their purity and identity using MALDI-TOF mass spectrometry. The purified DNA conjugates were lyophilized and stored at −20° C. until further use.

### Mass Characterization of BG-DNA Conjugates Using MALDI-TOF Mass Spectrometry

All purified products were characterized on an AB SCIEX 4800 MALDI TOF/TOF in the positive ion mode, with 3-Hydropicolinic acid (HPA) as the matrix. Samples were spotted onto a MALDI plate using a sandwich technique (sample-matrix-sample).

nbGFP Synthesis

The nbGFP protein was expressed from the recombinant plasmid, pBiEX1-nbGFP, which was a kind gift from Dr. Sivaraj Sivaramakrishnan (University of Minnesota, Twin Cities, USA). The SNAP-tagged protein construct contained, from the N- to C-terminus: the GFP nanobody (nbGFP), the SNAP-tag for oligo labeling, and both FLAG and 6×His tags for purification.

The plasmid was transformed into BL21 (DE3) competent E. coli (New England Biolabs), and a single colony of transformed cells was picked from LB-agar plates and used to inoculate a 50 mL culture in LB broth containing carbenicillin (100 ag/mL) antibiotics. This culture was grown for 16 hours at 37° C., and 250 rpm, at which point it was used to inoculate a 500 mL culture in LB supplemented with carbenicillin at the above concentration. The optical density of the culture was monitored until an OD₆₀₀ of 0.6-0.8 was reached. It was followed by gene induction using 0.5 mM IPTG for 16 hours at 220 rpm and 18° C. Cells were harvested via centrifugation at 3000×g for 15 minutes at 4° C. The supernatant was discarded, and the pellet was resuspended in 50 mL of lysis buffer (100 mM NaCl, 25 mM Tris at pH 8.0, 5 mM EDTA, 1% Triton-X, 1 mM DTT, and IX cOmplete protease inhibitor (Roche)) for 1 hour at −80° C. After thawing the lysate in RT, it was treated with hen egg white lysozyme (HEWL; Sigma-Aldrich) and DNase I (Sigma-Aldrich), each at a concentration of 1 mg/mL, for 30 minutes at 37° C. The mixture was transferred to an ice bath and sonicated for 10 minutes (1 second on, 2 seconds off, 50% amplitude). The lysate was centrifuged at 20000×g for 30 minutes at RT to separate cell debris from the periplasmic fraction.

The supernatant containing the nbGFP was loaded directly onto the HisTrap FF (Cytiva) 5 mL column equilibrated with Nickel Wash Buffer containing 25 mM Tris at pH 7.6, 500 mM NaCl, and 10 mM imidazole. To remove nonspecifically bound proteins, the resin was washed with 15 column volumes (CV) of the wash buffer, and the bound proteins were subsequently eluted with buffer containing 25 mM Tris at pH 7.6, 150 mM NaCl, and 500 mM imidazole. The eluted fractions were run on a 15% SDS-PAGE gel to confirm protein expression. The fractions mainly containing pure proteins with 35 kDa bands were pooled together and buffer exchanged using a 3.5 kDa MWCO centrifugal filter unit (Amicon) into an anion exchange buffer containing 20 mM Tris at pH 8.0, 10 mM NaCl. The protein solution was then injected into a HiTrap Q FF anion exchange 5 mL column (Cytiva), equilibrated with the anion exchange buffer, and finally eluted using a buffer containing 20 mM Tris at pH 8.0 and 500 mM NaCl. Nanobody constructs were buffer exchanged into PBS using a 3.5 k MWCO centrifugal filter unit, divided into aliquots that were flash-frozen in liquid nitrogen and kept at −80° C. until further use.

### ELISA Assay

96-well Nunc MaxiSorp flat bottom ELISA plates were coated with 100 μL of PRV 486 at 1×10⁹ particles/mL, diluted in ELISA coating buffer, and incubated overnight at 4° C. All the wash steps were performed thrice with 200 μL of IX Tris Buffered Saline+0.05% Tween20 (TBST), each wash lasting 5 minutes. Wells without virus coating were used as negative controls. Wells with EGFP coating were used as positive controls. After incubation, the plates were washed and blocked with 200 μL of 1 mg/mL casein in TBST for 2 hours at room temperature. Plates were washed, after which 100 μL of nbGFP or nbGFP conjugates or GFP aptamers were added in different concentrations after dilution in TBST+0.1% BSA and incubated for 1 hour at room temperature. Rabbit anti-pHluorin antibodies were used as positive controls for nbGFP. Plates were washed, and 100 μL of 1:10000 dilution of anti-His Horseradish peroxidase (HRP) for nbGFP, 100 μL of 1:10000 dilution of anti-rabbit HRP for anti-GFP antibodies, and 100 μL of 1:10000 dilution of streptavidin-HRP for GFP aptamers were added and incubated for 1 hour at room temperature. Plates were washed, and 100 μL of 3,3′,5,5′-tetramethylbenzidine (TMB) substrate was added and incubated in the dark for 2-3 minutes at room temperature. The reaction was quenched with 100 μL of 0.15 M H₂SO₄, and absorbance was read immediately at 450 nm using a microplate reader (Spectra MAX 190, Molecular Devices, Inc.).

### Flow Cytometry-Based Neutralization Assay

PK15 cells were seeded a day before in 24-well cell culture plates at a density of 7.5×10⁴ cells per well. The cells reached 70-80% confluency at this seeding density the next day. Approximately 1.5×10⁴ infectious units of PRV 486 were incubated with different concentrations of SC-nbGFP in a final volume of 100 μL for 1 hour at 37° C. Fresh DMEM was used to dilute stocks of SC-nbGFP constructs and to make up the final reaction volume. Before infection, the media from 24-well plates was removed, and the cells were washed once with IX PBS. The virus mixtures were added to the individual wells, and the cells were incubated to allow virus adsorption for 1 hour in a humidified 37° C., 5% CO₂ incubator. After 1 hour, 400 μL of virus medium was added to the wells to make up the final volume of 500 μL per well.

After 48 hours, cells were harvested for flow cytometry. The cell supernatant was removed from the wells, and the cells were washed once with PBS. Then the cells were fixed with 100 μL of 4% paraformaldehyde (PFA) for 20 minutes at room temperature with gentle shaking. Cells were washed and dissociated with 100 μL of 0.15% trypsin for 5 minutes at room temperature with gentle shaking. The trypsin was inactivated by adding 200 PL of PBS+2% FBS. Plates were centrifuged at 300×g for 5 minutes. The supernatant was discarded, and the cell pellets were reconstituted in 200 μL of PBS+2% FBS and transferred to individual wells in 96-well flow cytometry round bottom plates. Samples were acquired and analyzed using an Attune N×T flow Cytometer and software (Thermo Fisher), respectively. In total, 3×10⁴ single-cell events, gated on side scatter area versus height, were recorded for analysis. EGFP was excited with a 488 nm laser, and emission was measured with a 530/30 nm bandpass filter. Untreated cells were used as negative controls, and cells treated only with PRV 486 were used as positive controls. The residual infectivity (%) was calculated using the control group with the virus as the reference. Nonlinear regression for dose-response: inhibition was used to curve fit the data and analyze the half-maximal inhibitory concentration dosage (IC₅₀).

### Cytotoxicity Assay

Cytotoxicity resulting from treatment with SCnbGFP constructs or individual components was analyzed using the Promega LDH kit. In brief, PK15 cells were seeded in a 96-well cell culture plate a day before at a density of 50000 cells per well. To remove residual LDH activity from the cells, the overnight media was replaced with 100 μL of fresh media. SC60H-nbGFP, SC60H, nbGFP, and M13mp18 constructs were diluted to different concentrations in DMEM and added to the wells at 50 μL per well, for a total of 150 μL. Cells were incubated for 24 hours in a humidified incubator at 37° C. and 5% CO₂ Untreated cells were used as negative controls. Cells treated with the lysis buffer were used as positive controls and as a reference to calculate the cell viability of other groups. After incubation, the cell supernatant was removed and carefully transferred into individual wells of an optically clear 96-well flat-bottom microplate. A 50 μL LDH reaction mixture was added to the wells, and the plate was incubated in the dark for 30 minutes at room temperature. To stop the reaction, 50 μL of the stop solution was added to each well, and the absorbance was read within one hour using a microplate reader (Spectra MAX 190, Molecular Devices, Inc.). Cytotoxicity was calculated according to the manufacturer's protocol, and cell viability was calculated as 1-cytotoxicity.

### Serum Stability Assay

The stability of the conjugated snub cube nanostructures was evaluated in vitro by incubation at 37° C. for periods of 0, 1, 2, and 8 hours. 20 μL reactions containing 5 nM of the conjugated snub cube and DMEM supplemented with 0, 2, or 10% FBS were incubated for the respective duration before analysis with agarose gel electrophoresis. 2 μL of the sample were combined with 3 μL of water and 1 μL of 6× loading dye and loaded into a 1% agarose gel pre-stained with 1×SYBR GOLD. The samples were run for 1 hour in a running buffer of 1×TAE+12.5 mM MgCl₂ at 100V before visualization with a Bio-Rad Molecular Imager Gel Doc XR System transilluminator at the SYBR GOLD excitation wavelength (495 nm).

### Particle Size Distribution Analyses

NanoSight assays were performed to characterize the particle size distributions of complexes formed by the interactions between PRV 486 and SC601H-nbGFP. Nanoparticle tracking analysis (NTA) measurements were performed using a NanoSight NS300 instrument (Malvern Panalytical Ltd.), following the manufacturer's instructions. The virus samples with or without SC60H-nbGFP were serially diluted with PBS to reach a particle concentration of 10⁷-10⁹ particles/mL, suitable for NTA. The samples were injected into the sample unit with 1 ml, Luer-Slip sterile syringes (VWR). The capture settings (shutter and gain) and analysis settings were manually set. Each group was run in at least two different sample dilutions, and each sample was analyzed thrice. The video was recorded for 60 seconds at 30 fps for each measurement and analyzed using Nanoparticle Tracking Analysis (NTA) 2.0 Analytical software.

To roughly estimate the size of SC60H-nbGFP, a dynamic light scattering (DLS) analysis was performed. DLS measurements were acquired on a Zetasizer instrument (Malverm Panalytical Ltd.). The SC60H-nbGFP samples were diluted in TAE buffer containing 12.5 mM MgCl₂, to final concentrations of 50 μM, 500 μM, and 5 nM. 1 mL sample volume was loaded into a glass cuvette. Each sample was analyzed twice, and the size distributions of samples with an acceptable polydispersity index (PDI) were considered.

### Widefield Microscopy

For widefield fluorescence microscopy experiments, PK15 cells were seeded a day before in a p-slide 8-well plate (Ibidi) at a density of 1′10⁴ cells per well. Approximately 10⁸ infectious units of PRV 483 were mixed with 25 nM of SC60H-nbGFP in a final 100 μL volume and incubated at 37° C. for 1 hour. 30 minutes before imaging, cells were washed and incubated with Hoechst solution at 1 μM final concentration. The Hoechst solution was removed, and the virus and inhibitor cocktails were added to the cells. Cells were imaged on a Nikon Ti2-E inverted fluorescence microscope using three wavelengths 405 (nucleus blue), 488 (virus envelope, green), and 555 (virus capsid, red). Z-stacks consisted of ~10 images per stack, spaced by 0.2 m, and 4 to 5 fields of view were acquired for each sample. Image analyses were performed using Mathematica software.

### Image Analysis

Images were analyzed using custom Mathematica code. The Blue channel used to image the Hoechst stain was used to manually create a mask of the entire cell boundary. Z-stacks of the red and green channels were manually selected to encompass images containing cell periphery and those selected images were max projected. The mask was applied to the max projected images. A custom peak finder similar was used to identify individual puncta in both the green and red channels. Colocalization events of green and red puncta were determined when the centroid distance between individual puncta was within 10 pixels.

### Statistical Analysis

Analyses were performed with GraphiPad Prism 9 with measurements taken from distinct samples. The p values were computed by unpaired two-tailed Student's t tests. All reported measurements are expressed as mean±standard deviation (SD). Significant differences were detected when P<0.05.

### Example 3

### Exemplary Sequences

Exemplary sequences for various components of the multivalent antiviral molecules described herein are provided in the tables below.

SUPPLEMENTARY TABLE 1
Sequences of SC60H

| Sequence | |
|---|---|
| position | Sequence (5′ to 3′) |
| 1--snub-- | AGA CTT TTT TTC AAA TAT CGC GGA AGC AAA CTT TTT |
| | TCC AAC (SEQ ID NO: 1) |
| 2--snub-- | AGG TCG AAC CAT CAC CTT TTC AAA TCA AGT TCG GA |
| | (SEQ ID NO: 2) |
| 3--snub-- | ACG TCA TTT TAA GGG CGA AAT GAT AAG AGG TTT TTT |
| | CAT TTT TGC GCT GTA (SEQ ID NO: 3) |
| 4--(sh)snub_ | TTT TTT TTT TTT TTT TTT TTT TCA CTA CGT AGG ATT AGA |
| V3-- | GAG TAC CTT TAA CAG GGC GAT GGC C (SEQ ID NO: 4) |
| 5--snub-- | TTG CTC CTT TAA CCG TCT AT (SEQ ID NO: 5) |
| 6--snub-- | TGA TTC CAT TAG ATA CTT TTT ATT TCG CAA ACT CCA |
| | (SEQ ID NO: 6) |
| 7--snub-- | AGT GTT TTT TGT TCC AGT TTA GTG CCA AGC TTT TTT |
| | TGC AT (SEQ ID NO: 7) |
| 8--(sh)snub_ | TTT TTT TTT TTT TTT TTT TTT TAA CGT GGA TGG TCA ATA |
| V3-- | ACG ACG TTG TAA TCC ACT ATT AAA G (SEQ ID NO: 8) |
| 9--snub-- | AAC GAC GGC CGG AAC AAG AG (SEQ ID NO: 9) |
| 10--snub-- | AAT TCC TTT TTA CAC AAC ATA TGC CTA ATG AGT TTT |
| | TTG AGC TAA CTG GTT G (SEQ ID NO: 10) |
| 11--snub-- | CGA AAT TTT TCG GCA AAA TCT TCC AGT CGG GTT TTT |
| | AAA CC (SEQ ID NO: 11) |
| 12--(sh)snub_ | TTT TTT TTT TTT TTT TTT TTT TCG AGA TAG CAC ATT AAT |
| V3-- | TGC GTT GCG CTC CAA AAG AAT AGC C (SEQ ID NO: 12) |
| 13--snub-- | ACT GCC CGC TCC TTA TAA AT (SEQ ID NO: 13) |
| 14--snub-- | ACC CTA TTT TTA AGG GAG CCC GAC TAT TAT AGT TTT |
| | TTC AGA AGC AAC CGA A (SEQ ID NO: 14) |
| 15--snub_V3-- | GGC GAA AGC CGT AAA GCA CTA AAT TTT TGG GGT CG |
| | (SEQ ID NO: 15) |
| 16--snub-- | GGG CAA TTT TTC AGC TGA TTG CAA GCG GTC CAT TTT |
| | TCG CTG GTT TGG GTT C (SEQ ID NO: 16) |
| 17--(sh)snub_ | TTT TTT TTT TTT TTT TTT TTT TAG GTA TCC TGT TTG ATG |
| V3-- | GTC CCC AGC A (SEQ ID NO: 17) |
| 18--snub-- | TGG ATT TTT TTA TTT ACA TTG AAA GGG ACA TTT TTT |
| | TCT GGC CAA CAA GAA T (SEQ ID NO: 18) |
| 19--snub-- | ACG TGG TTT TTC ACA GAC AAT TTA CCT TTT TTT TTT |
| | TAA TGG (SEQ ID NO: 19) |
| 20--(th)snub_ | TTT TTT TTT TTT TTT TTT TTT TAC GTG ACC TGA AAG CGT |
| V3-- | AGA GAT AGA (SEQ ID NO: 20) |
| 21--snub_V3-- | ACC CTT CAG TAG ATT TAG TTT GAC CCA ATT CTG CGA |
| | (SEQ ID NO: 21) |
| 22--snub_V3-- | GCT CAA TTT TTC ATG TTT TAA CAT TCC ATA TAT TTT |
| | TAC AGT (SEQ ID NO: 22) |
| 23--(th)snub_ | TTT TTT TTT TTT TTT TTT TTT TTA CCG GTG TCT GGA AGT |
| V3-- | TTA TAT GCA A (SEQ ID NO: 23) |
| 24--snub_V3-- | CTA AAG TAC GAA CGA ACC ACC AGC GCC ATT AAA AA |
| | (SEQ ID NO: 24) |
| 25--snub_V3-- | AAA CAT TTT TTC AAG AAA ACA GAA CTG ATA GCT TTT |
| | TCC TAA AAC ATC AGA A (SEQ ID NO: 25) |
| 26--snub_V3-- | GAT AAA TTT TTA CAG AGG TGA ATC GGG AGA AAT TTT |
| | TCA ATA (SEQ ID NO: 26) |
| 27--snub_V3-- | AAT CTA AAT TGC TGA ATA TAA TGG ATG GCT TAG A |
| | (SEQ ID NO: 27) |
| 28--(th)snub_ | TTT TTT TTT TTT TTT TTT TTT TGC TTA AGC ATC ACC TTG |
| V3-- | CTG AAA TGA AA (SEQ ID NO: 28) |
| 29--snub-- | GCT GAG TTT TTA GCC AGC AGC AAC CTC AAA TAT TTT |
| | TTC AAA (SEQ ID NO: 29) |
| 30--snub-- | GCC TGG CTC GAA TTC GTT TTT TAA TCA TGG TCT CAC |
| | (SEQ ID NO: 30) |
| 31--snub_V3-- | ACC AGT CGA TCC CCG GGT ACC GAC AGG TCG ACT C |
| | (SEQ ID NO: 31) |
| 32--(th)snub_ | TTT TTT TTT TTT TTT TTT TTT TTA GAG ACA CGA CCA |
| V3-- | GTA ATA GCA GAT TC (SEQ ID NO: 32) |
| 33--snub_V3-- | GAA CAA TGT GAA ATT GTT ATC CGC ATA GCT GTT TC |
| | (SEQ ID NO: 33) |
| 34--snub-- | CTA TAT TTT TTG TAA ATG CTG CAA ACT ATC GGT TTT |
| | TCC TTG CTG GTT GCA A (SEQ ID NO: 34) |
| 35--snub-- | CAG GAA TTT TTA AAC GCT CAT ACA TAG CGA TAT TTT |
| | TGC TTA (SEQ ID NO: 35) |
| 36--(th)snub_ | TTT TTT TTT TTT TTT TTT TTT TCT GTA TTA CCG CCA GCC |
| V3-- | ATA ATA TCC A (SEQ ID NO: 36) |
| 37--snub_V3-- | CCA TCA CAG TGT AAA GCC TGG GGC GAG CCG GAA GCA |
| | (SEQ ID NO: 37) |
| 38--snub-- | AGG CCA TTT TTC CGA GTA AAA TAG CAA TAC TTT TTT |
| | TCT TTG (SEQ ID NO: 38) |
| 39--(th)snub_ | TTT TTT TTT TTT TTT TTT TTT TTA AGC AAA TTA ACC GTT |
| V3-- | GGA GTC TGT (SEQ ID NO: 39) |
| 40--snub-- | GTC TTT ACC CTC CGA TTT AGA G (SEQ ID NO: 40) |
| 41--(th)snub_ | TTT TTT TTT TTT TTT TTT TTT TAA GCC GGC GGA CCA |
| V3-- | TAA ATC AAA AAT CAG CTT GAC GGG GA (SEQ ID NO: 41) |
| 42--snub-- | TTC AGA TTT TTA AAC GAG AAT AAC GTG GCG AGT TTT |
| | TAA AGG (SEQ ID NO: 42) |
| 43--snub_V3-- | TAC ATT TAA GAT TAA GAG GAA GCA GCG GAT TGC AT |
| | (SEQ ID NO: 43) |
| 44--snub-- | ATA TAA TTT TTT CCT GAT TGT ACT AAT AGA TTT TTT |
| | TAG AGC CGT CAT TAG A (SEQ ID NO: 44) |
| 45--snub-- | CTT TAC TTT TTA AAC AAT TCG TTA TTA ATT TTT TTT |
| | TAA AAG (SEQ ID NO: 45) |
| 46--(th)snub_ | TTT TTT TTT TTT TTT TTT TTT TCA AAG AGG ATT TAG |
| V3-- | AAG TAA TAG ATA A (SEQ ID NO: 46) |
| 47--(th)snub_ | TTT TTT TTT TTT TTT TTT TTT TTC CAA AGC GAA CCA |
| V3-- | GAC CGT TTT AAT (SEQ ID NO: 47) |
| 48--snub_V3-- | TCG AGC TTA GTT GGC AAA TCA ACA ATC AAT ATC TGG |
| | (SEQ ID NO: 48) |
| 49--snub_V3-- | CCC TCA GTT GAA AGG ATT TTT ATT GAG GAA GAG AAC |
| | (SEQ ID NO: 49) |
| 50--snub-- | TGT CGG GGG AGA GGC GTT TTT GTT TGC GTA TGA GAC |
| | (SEQ ID NO: 50) |
| 51--(th)snub_ | TTT TTT TTT TTT TTT TTT TTT TGA GGA ATC GGC CAA |
| V3-- | CGC GCT GCC AGC T (SEQ ID NO: 51) |
| 52--snub_V3-- | GCA TTA ATA AGT GTT TTT ATA ATC GCC AGA ATC CT |
| | (SEQ ID NO: 52) |
| 53--snub_V3-- | TTG AAA TTT TTT ACC GAC CGT GGA TTT TAG ACT TTT |
| | TAG GAA CGG TAC AGT G (SEQ ID NO: 53) |
| 54--snub_V3-- | TGC TTT GTT TTC TTT TCA CCA GTT GGG CGC CAG GG |
| | (SEQ ID NO: 54) |
| 55--snub-- | TTT AAC TTT TTA ACG CCA ACA ATG CGC CGC TAT TTT |
| | TCA GGG CGC GTG TGC T (SEQ ID NO: 55) |
| 56--snub-- | TTC CTC TTT TTG TTA GAA TCA ACC GGA ATC ATT TTT |
| | TAA TTA (SEQ ID NO: 56) |
| 57--(th)snub_ | TTT TTT TTT TTT TTT TTT TTT TTG GTA CGA GCA CGT ATA |
| V3-- | ACA CTA TGG T (SEQ ID NO: 57) |
| 58--snub_V3-- | GAA AGG ACC TGA GAG AGT TGC AGC CCT TCA CCG CC |
| | (SEQ ID NO: 58) |
| 59--snub-- | AAG GGC TGG CAA GTG TTT TTT AGC GGT CAC GTA ATA |
| | (SEQ ID NO: 59) |
| 60--(th)snub_ | TTT TTT TTT TTT TTT TTT TTT TTG GCG CGG GCG CTA |
| V3-- | GGG CGA AGA AAG C (SEQ ID NO: 60) |
| 61--snub-- | TTC ATT TGA AAT TTT TGA AT (SEQ ID NO: 61) |
| 62--(th)snub_ | TTT TTT TTT TTT TTT TTT TTT TTT TAA CAA TGG CTA TTA |
| V3-- | GTC TTT AAT GCG CAA ATT AAT TAC A (SEQ ID NO: 62) |
| 63--snub-- | AAA CAT TGC TTC TGT ATT TTT AAT CGT CGC TTG AAA |
| | (SEQ ID NO: 63) |
| 64--snub-- | ATC CTT GAA AGG AAA TAC CT (SEQ ID NO: 64) |
| 65--(sh)snub_ | TTT TTT TTT TTT TTT TTT TTT TCC CTT AGA ACA TTT TGA |
| V3-- | CGC TCA ATC GTC ATT AAT TAA TTT T (SEQ ID NO: 65) |
| 66--snub-- | AAA GAA CGC GTA ATA ACA TC (SEQ ID NO: 66) |
| 67--snub-- | ATT AGA GAA AAC TTT TTT TTT TCA AAT ATA TAT GGT |
| | (SEQ ID NO: 67) |
| 68--(th)snub_ | TTT TTT TTT TTT TTT TTT TTT TTC GCA AGA CAC TTG CCT |
| V3-- | GAG TAG AAG AAC TAT GCA AAT CCA A (SEQ ID NO: 68) |
| 69--(sh)snub_ | TTT TTT TTT TTT TTT TTT TTT TCG TTA AAT TAA ACA GGA |
| V3-- | GGC CGA TTA AAG GTG ATA AAT AAG G (SEQ ID NO: 69) |
| 70--snub-- | AAG AAT AAA CGA GCG GGA GC (SEQ ID NO: 70) |
| 71--snub-- | TTC GAG CCA GCT GCG CGT AA (SEQ ID NO: 71) |
| 72--snub-- | AGA GAA TTT TTT ATA AAG TAC CCA ATA CTG CGT TTT |
| | TGA ATC GTC ATA ACA G (SEQ ID NO: 72) |
| 73--(th)snub_ | TTT TTT TTT TTT TTT TTT TTT TGA GGC ATT CCA CCA CAC |
| V3-- | CCG CCG CGC TTA TGT AAT TTA GGC A (SEQ ID NO: 73) |
| 74--(sh)snub_ | TTT TTT TTT TTT TTT TTT TTT TAA TGC TTT AAA ATA TTC |
| V3-- | ATT GAA TCT CCT TTG C (SEQ ID NO: 74) |
| 75--snub_V3-- | CCG AAC GAC AAC TCG TAT TAA ACC CCT CA (SEQ ID NO: |
| | 75) |
| 76--snub-- | TGG AAG GGT TGT TAT CTA AA (SEQ ID NO: 76) |
| 77--snub-- | CTA CCA TTT TTT ATC AAA ATT GTA GAT TTT CAT TTT |
| | TGG TTT AAC GTG CCA C (SEQ ID NO: 77) |
| 78--(th)snub_ | TTT TTT TTT TTT TTT TTT TTT TTC TGA ATA AAT ATC TTT |
| V3-- | AGG AGC ACT AAC ATT GGA TTA TAC T (SEQ ID NO: 78) |
| 79--(sh)snub_ | TTT TTT TTT TTT TTT TTT TTT TTG CAA CAG TCA GAT |
| V3-- | GAA TAT ACA GTA ACA GAT TAA CAC CGC C (SEQ ID NO: |
| | 79) |
| 80--snub-- | TAC CTT TTA CGG CGG TCA GT (SEQ ID NO: 80) |
| 81--snub-- | AAT TGA TTT TTG TTA AGC CCA ATA GCT ATC TTT TTT |
| | TAC CGA AGC CCA GTT A (SEQ ID NO: 81) |
| 82--snub-- | CCA GAA TTT TTG GAA ACC GAG AAT AGG AAC CCT TTT |
| | TAT GTA (SEQ ID NO: 82) |
| 83--snub-- | CCG TAA ACG CCT GTA GTT TTT CAT TCC ACA GAC AAG |
| | (SEQ ID NO: 83) |
| 84--snub-- | TAA CAT TTT TTA AAA ACA GGG ACC CTG AAC AAT TTT |
| | TAG TCA (SEQ ID NO: 84) |
| 85--snub-- | GAG GGG TTT TGT CGT CTT TTT TTT CCA GAC GGC TAA |
| | (SEQ ID NO: 85) |
| 86--snub-- | ACA ACT TTT TTT TCA ACA GTT AGA AAC GAT TTT TTT |
| | TTT TGT TTA ACG AGA A (SEQ ID NO: 86) |
| 87--snub-- | TTT TAT TTT TTC CTG AAT CTT TAA TTT GCC AGT TTT TTT |
| | ACA AAA TAA AGG A (SEQ ID NO: 87) |
| 88--snub-- | ATT GCG TTT TTA ATA ATA ATT CTC CAA AAG GAT TTT |
| | TGC CTT (SEQ ID NO: 88) |
| 89--snub-- | TAA TTA ATT TCT TAA ATT TTT CAG CTT GAT ATA CAA |
| | (SEQ ID NO: 89) |
| 90--snub-- | AGG TTT TTT TTT GAA GCC TTA TCG CCC ACG CAT TTT |
| | TTA ACC GAT ATA GGC C (SEQ ID NO: 90) |
| 91--snub-- | GCT TTT TTT TTG CGG GAT CGT CAT CGT AGG AAT TTT |
| | TTC ATT (SEQ ID NO: 91) |
| 92--snub-- | AAA CCA TTT TTA TCA ATA ATC GTA TTA AAC CAT TTT |
| | TAG TAC CGC ACG AGG G (SEQ ID NO: 92) |
| 93--snub-- | TAG CAA TTT TTC GGC TAC AGA GAA GTT TCC ATT TTT |
| | TTA AAC (SEQ ID NO: 93) |
| 94--snub-- | GGG TAC CTA AAA CGA ATT TTT AGA GGC AAA ATG TAG |
| | (SEQ ID NO: 94) |
| 95--snub-- | AAT AAA TTT TTC AAC ATG TTC AAT AGA TAA GTT TTT |
| | TCC TGA (SEQ ID NO: 95) |
| 96--snub-- | ACA AGA GCG ATT ATA CTT TTT CAA GCG CGA ACT GAT |
| | (SEQ ID NO: 96) |
| 97--snub-- | AAA TTG TTT TTT GTC GAA ATC AAA GAA GTT TTT TTT |
| | TGC CAG AGG GGA CGA C (SEQ ID NO: 97) |
| 98--snub-- | CGT TTA TTT TTC CAG ACG ACG GAG GCG CAG ACT TTT |
| | TGG TCA (SEQ ID NO: 98) |
| 99--snub-- | ATC ATG GAC AGA TGA ATT TTT CGG TGT ACA GAA GAG |
| | (SEQ ID NO: 99) |
| 100--snub-- | TAA TCT TTT TTT GAC AAG AAC TAC ATA ACG CCT TTT |
| | TAA AAG GAA TTA CCC T (SEQ ID NO: 100) |
| 101--snub-- | AAT AAT AGA AAG ATT CTT TTT ATC AGT TGA GAG CTG |
| | (SEQ ID NO: 101) |
| 102--snub-- | CTC ATT TTT TTC AGT GAA TAA GTA GTA AAT TGT TTT |
| | TGG CTT GAG ATT ACC T (SEQ ID NO: 102) |
| 103--snub-- | TAT GCG TTT TTA TTT TAA GAA AAG AAA AAT CTT TTT |
| | TAC GTT (SEQ ID NO: 103) |
| 104--(sh)snub_ | TTT TTT TTT TTT TTT TTT TTT TCT CAG TAC GCA GCA CCG |
| V3-- | TAA TCA GTA GCG TAG CGG GGT TTT G (SEQ ID NO: 104) |
| 105--snub-- | AAT GAA TTT TTA CCA TCG ATA CAG GCG GAT AAT TTT |
| | GTG CC (SEQ ID NO: 105) |
| 106--snub-- | GTC GAA CAA AAG GGC GTT TTT ACA TTC AAC CAT TAT |
| | (SEQ ID NO: 106) |
| 107--snub-- | TCA TTA TTT TTA AGG TGA ATT TAG AGC CAG CAT TTT |
| | TAA ATC ACC AGT CAC C (SEQ ID NO: 107) |
| 109--(sh)snub_ | TTT TTT TTT TTT TTT TTT TTT TAG TAT AGC CCG GAA |
| V3-- | TAG GAG AAA ATT CAT ATG GTT TAC CAT A (SEQ ID NO: |
| | 108) |
| 108--snub-- | AGC GCC AAA GGA GGG TTG AT (SEQ ID NO: 109) |
| 110--snub-- | CAT AAA TTT TTG GTG GCA ACA GTT TAT TTT GTT TTT |
| | TCA CAA TCA ATT GTA T (SEQ ID NO: 110) |
| 111--snub-- | CAC CGT TTT TAC TCA GGA GGC AGA ACC GCC ATT TTT |
| | CCC TC (SEQ ID NO: 111) |
| 112--snub-- | AGA GCA CTG GCA TGA TTT TTT TAA GAC TCC TAC ATA |
| | (SEQ ID NO: 112) |
| 113--(sh)snub_ | TTT TTT TTT TTT TTT TTT TTT TGC CTC AGA ACC GCC ACC |
| V3-- | CTT TTA GTA C (SEQ ID NO: 113) |
| 114--snub_ | CGC CAC CCC GTA TAA ACA GTT AAT TGA GTA ACA GT |
| V3-- | (SEQ ID NO: 114) |
| 115--snub-- | TAC CCA AAA GAC ACC ACC CTC A (SEQ ID NO: 115) |
| 116--(th)snub_ | TTT TTT TTT TTT TTT TTT TTT TTA ACG GAA TTT TCA GGG |
| V3-- | ATA GCA AGC CCG AAA CGC AAT AA (SEQ ID NO: 116) |
| 117--(th)snub_ | TTT TTT TTT TTT TTT TTT TTT TCA GAC CAG TAC AAA |
| V3-- | CTA CAC ACT GAG (SEQ ID NO: 117) |
| 118--snub_ | TTT CGT CGA GTG TAC TGG TAA TAG CTT TTG ATG ATA |
| V3-- | (SEQ ID NO: 118) |
| 119--snub-- | GAG ATA ACC CAC AGC CCT CA (SEQ ID NO: 119) |
| 120--(th)snub_ | TTT TTT TTT TTT TTT TTT TTT TAA TAT CAG ATA GTT AGC |
| V3-- | GTA ACG ATC TAA ATA ATT GAG CGC T (SEQ ID NO: 120) |
| 121--snub_ | AAA GCG CAT CTG TAT GGG ATT TTT TAG TAA ATG AA |
| V3-- | (SEQ ID NO: 121) |
| 122--(th)snub_ | TTT TTT TTT TTT TTT TTT TTT TTT TGT CTC TGA ATT TAC |
| V3-- | CGC AGA ATG G (SEQ ID NO: 122) |
| 123--snub-- | AAT CCA AAT ATC AGC GGA GT (SEQ ID NO: 123) |
| 124--(sh)snub_ | TTT TTT TTT TTT TTT TTT TTT TTT TAT CCC GAG AAT AGA |
| V3-- | AAG GAA CAA CTA AAC AGC CAT ATT A (SEQ ID NO: 124) |
| 125--(th)snub_ | TTT TTT TTT TTT TTT TTT TTT TTT GGT CTC CAA AAA AAA |
| V3-- | GGT TTT CAC G (SEQ ID NO: 125) |
| 126--snub_ | TTG AAA ACC TTG ATA TTC ACA AAC AGG TCA GAC GA |
| V3-- | (SEQ ID NO: 126) |
| 127--snub_ | CGC CAC CCC TTG CTT TCG AGG TGG TAT CGG TTT A (SEQ |
| V3-- | ID NO: 127) |
| 128--(th)snub_ | TTT TTT TTT TTT TTT TTT TTT TTC AGT CAG AAC CGC CAC |
| V3-- | CCT CTC AGA GC (SEQ ID NO: 128) |
| 129--snub-- | TGC ACC CAG CCC GAT AGT TG (SEQ ID NO: 129) |
| 130--(th)snub_ | TTT TTT TTT TTT TTT TTT TTT TCC GAC AAT GAC AAC |
| V3-- | AAC CAA ATC AAG ATT AGT TGC TAT TTC G (SEQ ID NO: |
| | 130) |
| 131--snub_ | ATA ATC AAC TTG CAG GGA GTT AAA TTC GGT CGC TG |
| V3-- | (SEQ ID NO: 131) |
| 132--(th)snub_ | TTT TTT TTT TTT TTT TTT TTT TAG GAA TCA CCG GAA |
| V3-- | CCA GAA TCT TTT C (SEQ ID NO: 132) |
| 133--snub-- | TTT TTA TTT TCA CCC TCA GC (SEQ ID NO: 133) |
| 134--(sh)snub_ | TTT TTT TTT TTT TTT TTT TTT TAT CGG AAC TCA TCG AGA |
| V3-- | ACA AGC AAG CCG AGC GAA AGA CAG C (SEQ ID NO: 134) |
| 135--snub_ | TTT TCA TCA GAC TTT TTC ATG AGG GCT TTG AGG AC |
| V3-- | (SEQ ID NO: 135) |
| 136--(th)snub_ | TTT TTT TTT TTT TTT TTT TTT TTA AGG CAT TTT CGG TCA |
| V3-- | TAG TAG CGC G (SEQ ID NO: 136) |
| 137--(th)snub_ | TTT TTT TTT TTT TTT TTT TTT TCA TTC TAC GAA GGC ACC |
| V3-- | AAA AAT ACG T (SEQ ID NO: 137) |
| 138--snub_ | AAT GCC AAG CAA GGC CGG AAA CGT AGC ACC ATT AC |
| V3-- | (SEQ ID NO: 138) |
| 139--snub-- | TTT ACG AGC AGA ATA CAC TA (SEQ ID NO: 139) |
| 140--(th)snub_ | TTT TTT TTT TTT TTT TTT TTT TCC ATC CTA AAA ACA CTC |
| V3-- | ATC TTT GAC CCC CAA AAA TAA TAT C (SEQ ID NO: 140) |
| 141--(th)snub_ | TTT TTT TTT TTT TTT TTT TTT TGA CTG ATT TGT ATC ATC |
| V3-- | GCA CAA AGT A (SEQ ID NO: 141) |
| 142--snub_ | CAA CGG ATG AGC CAT TTG GGA ATA TCA CCG TCA CC |
| V3-- | (SEQ ID NO: 142) |
| 143--snub-- | GGC TTT TGC ACG CGA CCT GC (SEQ ID NO: 143) |
| 144--(sh)snub_ | TTT TTT TTT TTT TTT TTT TTT TAT AGC GAG ATC CAT GTT |
| V3-- | ACT TAG CCG GAA CAT AAA AAC CAA A (SEQ ID NO: 144) |
| 145--snub_ | GAG GGA AGA CCA ACT TTG AAA GAA AGG GAA CCG AAC |
| V3-- | (SEQ ID NO: 145) |
| 146--(th)snub_ | TTT TTT TTT TTT TTT TTT TTT TTG GTA AAT ATT GAC GGA |
| V3-- | AGA TTG AGG (SEQ ID NO: 146) |
| 147--snub_ | AAA CGC AAT GGC TGA CCT TCA TCA CCA GGC GCA TA |
| V3-- | (SEQ ID NO: 147) |
| 148--(th)snub_ | TTT TTT TTT TTT TTT TTT TTT TGG CAG ACA CCA CGG |
| V3-- | AAT AAT ATA AAA G (SEQ ID NO: 148) |
| 149--snub-- | CTA ATG CAG ACG GAT ATT CA (SEQ ID NO: 149) |
| 150--(th)snub_ | TTT TTT TTT TTT TTT TTT TTT TCG TAA CAA ATT TAG GAA |
| V3-- | TAC CAC ATT CAA TTA CCC AAA TCA A (SEQ ID NO: 150) |
| 151--(th)snub_ | TTT TTT TTT TTT TTT TTT TTT TTG TTG AAA CAC CAG AAC |
| V3-- | GAG GCT TGC C (SEQ ID NO: 151) |
| 152--snub_ | CTG ACG AAG CAA ACG TAG AAA ATT ATT ACG CAG TA |
| V3-- | (SEQ ID NO: 152) |
| 153--(sh)snub_ | TTT TTT TTT TTT TTT TTT TTT TTG TGA ATG GTT TAA TTT |
| V3-- | CAA CTG CAG ATA G (SEQ ID NO: 153) |
| 154--snub-- | CCG AAC AAT TTT TAA GAA AAG TAA TTA ATC AT (SEQ ID |
| | NO: 154) |
| 155--snub-- | ACA GAA TCA AGG ATT AGG AT (SEQ ID NO: 155) |
| 156--snub-- | GAG AAG TTT GCC TTT ATT TTT GCG TCA GAC TGC CCC |
| | (SEQ ID NO: 156) |
| 157--snub-- | CTT ATT TTT TTA GCG TTT GCC GCC ACC ACC GGT TTT |
| | TAA CCG CCT CCC AGA G (SEQ ID NO: 157) |
| 158--snub-- | CCA CCA TTT TTC CCT CAG AGC GGC TGA GAC TCT TTT |
| | CTC AA (SEQ ID NO: 158) |
| 159--(sh)snub_ | TTT TTT TTT TTT TTT TTT TTT TTA TTA AGA CGC CAC CAG |
| V3-- | AAC CAC CAC CAG TGA AAC ATG AAA G (SEQ ID NO: 159) |
| 160--snub-- | AGC CGC CGC CCC TAT TAT TC (SEQ ID NO: 160) |
| 161--snub-- | GTG CCT GCC CCC TGC CTT TTT ATT TCG GAA AGC AT |
| | (SEQ ID NO: 161) |
| 162--snub-- | TGA CAG TTT TTG AGG TTG AGG CAA ATA AAT CCT TTT |
| | TTC ATT AAA GCT TCC A (SEQ ID NO: 162) |
| 163--snub-- | GTA AGC TTT TTG TCA TAC ATG AGT TTT AAC GGT TTT |
| | TGG TCA (SEQ ID NO: 163) |
| 164--(sh)snub_ | TTT TTT TTT TTT TTT TTT TTT TAC CGA AAT AAA GAA |
| V3-- | ATT GCA TTT GCA C (SEQ ID NO: 164) |
| 165--snub_ | GTA AAA CAA GTC AGG ACG TTG GGC TGG CTC ATT AT |
| V3-- | (SEQ ID NO: 165) |
| 166--snub-- | GAT TAA AAA TCA TAG GTT TTT TCT GAG AGA CTA TAA |
| | (SEQ ID NO: 166) |
| 167--snub_ | GTT TTA GGG CTT AGG TTG GGT TAT ACC TTT TTA AC |
| V3-- | (SEQ ID NO: 167) |
| 168--snub-- | ACC GCG CTT ATC CGG TTT TTT ATT CTA AGA AGC GGG |
| | (SEQ ID NO: 168) |
| 169--(th)snub_ | TTT TTT TTT TTT TTT TTT TTT TCT CCC GAA CCT CCC GAC |
| V3-- | TTC GCG AGG C (SEQ ID NO: 169) |
| 170--snub-- | CTA GAA ATT CTT ACC ATT TTT GTA TAA AGC CCC ATA |
| | (SEQ ID NO: 170) |
| 171--(th)snub_ | TTT TTT TTT TTT TTT TTT TTT TAA CGG CTT AAT TGA GAA |
| V3-- | TCG AAC GCT CA (SEQ ID NO: 171) |
| 172--snub_ | ACA GTA GGC GCC TGT TTA TCA ACA GCT AAT GCA G |
| V3-- | (SEQ ID NO: 172) |
| 173--snub-- | TTT GAA CCA GAA GGA GTT TTT CGG AAT TAT CCA TCA |
| | (SEQ ID NO: 173) |
| 174--snub_ | AAC GGA ATC AGA TGA TGG CAA TTA TCA TAT TCC TG |
| V3-- | (SEQ ID NO: 174) |
| 175--(th)snub_ | TTT TTT TTT TTT TTT TTT TTT TAT TAC AAC ATT ATT ACA |
| V3-- | GGA ACG AAC T (SEQ ID NO: 175) |
| 176--snub-- | ACG GAA ATC GCG CAG ATT TTT GGC GAA TTA TGA AAC |
| | (SEQ ID NO: 176) |
| 177--snub_ | TAG ACG GAG CAA AAG AAG ATG ATT CAT TTC AAT TAC |
| V3-- | (SEQ ID NO: 177) |
| 178--(th)snub_ | TTT TTT TTT TTT TTT TTT TTT TCT GGA GAA TTA ACT GAA |
| V3-- | CAA GCG CAT (SEQ ID NO: 178) |
| 179--snub_ | ATC AAT ATA GCA GCC TTT ACA GAG TCA AAA ATG AA |
| V3-- | (SEQ ID NO: 179) |
| 180--(sh)snub_ | TTT TTT TTT TTT TTT TTT TTT TAA TAT GTG AGT GAA TAA |
| V3-- | CCG TAC ATA A (SEQ ID NO: 180) |
| 181--(sh)snub_ | TTT TTT TTT TTT TTT TTT TTT TCA TTC TGA CCT AAA TTT |
| V3-- | ATT TAG TTA (SEQ ID NO: 181) |
| 182--snub_ | ATTTCATCAATCAGATATAGAAGGCCCAATAGCAAG (SEQ |
| V3-- | ID NO: 182) |
| 183--snub-- | TGG ATA GCG TCG ACA AAA GG (SEQ ID NO: 183) |
| 184--(sh)snub_ | TTT TTT TTT TTT TTT TTT TTT TTC CAG ACG GTA ATA GTA |
| V3-- | AAA TGT TTA GAC TAA AGT AAT TCT G (SEQ ID NO: 184) |
| 185--snub_ | ATCATTTTAGCAACACTATCATAACGAGGCATAG (SEQ ID |
| V3-- | NO: 185) |
| 186--(sh)snub_ | TTT TTT TTT TTT TTT TTT TTT TTA AGG CGG AAC AAA |
| V3-- | GAA ACC GTA ACA TT (SEQ ID NO: 186) |
| 187--snub_ | ATTGCTTTAACAATGAAATAGCAATAATAAGAGCA (SEQ ID |
| V3-- | NO: 187) |
| 188--(sh)snub_ | TTT TTT TTT TTT TTT TTT TTT TAG AGA ATA CCA AGT |
| V3-- | TAC AAT TCG CCT G (SEQ ID NO: 188) |
| 189--(sh)snub_ | TTT TTT TTT TTT TTT TTT TTT TGA GCA ATA GTG AAT TTA |
| V3-- | TCA GAC GCT G (SEQ ID NO: 189) |
| 190--snub_ | AGAAGAGTCGTCTTTCCAGAGCCACCAACGCTAAC (SEQ ID |
| V3-- | NO: 190) |
| 191--snub_ | TTCCTTATCATATGCGTTATACAAAAAGCCTGTTT (SEQ ID |
| V3-- | NO: 191) |
| 192--(sh)snub_ | TTT TTT TTT TTT TTT TTT TTT TAG TAT CAT TCC AAG AAC |
| V3-- | GGG GCT GTC T (SEQ ID NO: 192) |

TABLE 2
Handle sequence for SC1H

| S.N. | Position | Sequence |
|---|---|---|
| 1 | 12 | TTTTTTTTTTTTTTTTTTTTCGAG |
| | | ATAGCACATTAATTGCGTTGCGCT |
| | | CCAAAAGAATAGCC |
| | | (SEQ ID NO: 193) |

TABLE 3
Handle sequences for SC12H

| S.N. | Position | Sequence |
|---|---|---|
| 1 | 4 | TTTTTTTTTTTTTTTTTTTTCACT |
| | | ACGTAGGATTAGAGAGTACCTTTA |
| | | ACAGGGCGATGGCC |
| | | (SEQ ID NO: 194) |
| 2 | 12 | TTTTTTTTTTTTTTTTTTTTCGAG |
| | | ATAGCACATTAATTGCGTTGCGCT |
| | | CCAAAAGAATAGCC |
| | | (SEQ ID NO: 195) |
| 3 | 74 | TTTTTTTTTTTTTTTTTTTTAATG |
| | | CTTTAAAATATTCATTGAATCTCC |
| | | TTTGC (SEQ ID NO: 196) |
| 4 | 79 | TTTTTTTTTTTTTTTTTTTTTGCA |
| | | ACAGTCAGATGAATATACAGTAAC |
| | | AGATTAACACCGCC |
| | | (SEQ ID NO: 197) |
| 5 | 104 | TTTTTTTTTTTTTTTTTTTTCTCA |
| | | GTACGCAGCACCGTAATCAGTAGC |
| | | GTAGCGGGGTTTTG |
| | | (SEQ ID NO: 198) |
| 6 | 113 | TTTTTTTTTTTTTTTTTTTTGCCT |
| | | CAGAACCGCCACCCTTTTAGTAC |
| | | (SEQ ID NO: 199) |
| 7 | 144 | TTTTTTTTTTTTTTTTTTTTATAG |
| | | CGAGATCCATGTTACTTAGCCGGA |
| | | ACATAAAAACCAAA |
| | | (SEQ ID NO: 200) |
| 8 | 153 | TTTTTTTTTTTTTTTTTTTTTGTG |
| | | AATGGTTTAATTTCAACTGCAGAT |
| | | AG (SEQ ID NO: 201) |
| 9 | 180 | TTTTTTTTTTTTTTTTTTTTAATA |
| | | TGTGAGTGAATAACCGTACATAA |
| | | (SEQ ID NO: 202) |
| 10 | 181 | TTTTTTTTTTTTTTTTTTTTCATT |
| | | CTGACCTAAATTTATTTAGTTA |
| | | (SEQ ID NO: 203) |
| 11 | 189 | TTTTTTTTTTTTTTTTTTTTGAGC |
| | | AATAGTGAATTTATCAGACGCTG |
| | | (SEQ ID NO: 204) |
| 12 | 192 | TTTTTTTTTTTTTTTTTTTTAGTA |
| | | TCATTCCAAGAACGGGGCTGTCT |
| | | (SEQ ID NO: 205) |

Embodiments of the present disclosure are set forth in the following clauses:

Clause 1: A multivalent antiviral biomolecule comprising:
(i) a nucleic acid-based scaffold; and
(ii) a plurality of antiviral binders conjugated to the scaffold, wherein each antiviral binder of the plurality of binder targets one or more viral proteins, thereby neutralizing one or more viruses.

Clause 2: The biomolecule of clause 1, wherein the plurality of antiviral binders target the same viral protein.

Clause 3: The biomolecule of clause 1, wherein the plurality of antiviral binders target two or more distinct viral proteins.

Clause 4: The biomolecule of any one of clauses 1 to 4, wherein the nucleic acid-based scaffold comprises a DNA origami-based nanostructure.

Clause 5: The biomolecule of any one of clauses 1 to 3, wherein the nucleic acid-based scaffold comprises a DNA snub cube.

Clause 6: The biomolecule of clause 5, wherein the DNA snub cube comprises 24 vertices and 60 edges.

Clause 7: The biomolecule of any one of clauses 1 to 6, wherein at least one edge of the DNA snub cube is conjugated to at least one antiviral binder of the plurality of antiviral binders.

Clause 8: The biomolecule of any one of clauses 1 to 7, wherein the plurality of antiviral binders are conjugated to the nucleic acid-based scaffold via a linker.

Clause 9: The biomolecule of clause 8, wherein the linker comprises a single-stranded DNA overhang coupled to the scaffold.

Clause 10: The biomolecule of clause 9, wherein the linker comprises a single-stranded DNA complementary to the single-stranded DNA overhang.

Clause 11: The biomolecule of clause 10, wherein the complementary single-stranded DNA is conjugated to at least one antiviral binder of the plurality of antiviral binders.

Clause 12: The biomolecule of clause 11, wherein the complementary single-stranded DNA is conjugated to at least one antiviral binder of the plurality of antiviral binders via a SNAP-tag with a benzylguanine (BG) linker.

Clause 13: The biomolecule of any one of clauses 1 to 12, wherein at least one of the plurality of antiviral binders comprises a monoclonal antibody, a human antibody, a humanized antibody, a chimeric antibody, and/or a single-domain antibody.

Clause 14: The biomolecule of any one of clauses 1 to 13, wherein at least one of the plurality of antiviral binders comprises an antibody fragment selected from the group consisting of Fab, Fab-C, Fab′-SH, Fv, scFv, and (Fab′)₂ fragments.

Clause 15: The biomolecule of any one of clauses 1 to 14, wherein at least one of the plurality of antiviral binders comprises a single-domain antibody (nanobody).

Clause 16: The biomolecule of any one of clauses 1 to 12, wherein at least one of the plurality of antiviral binders comprises an aptamer.

Clause 17: The biomolecule of clause 16, wherein the aptamer is an RNA or DNA aptamer.

Clause 18: The biomolecule of clause 16 or clause 17, wherein the aptamer comprises a docking domain to facilitate conjugation to the scaffold.

Clause 19: The biomolecule of clause 18, wherein the aptamer further comprises a linker region between the docking domain and a region of the aptamer that binds a viral protein.

Clause 20: The biomolecule of clause 19, wherein the linker region is from about 2 to about 40 base pairs in length.

Clause 21: The biomolecule of any one of clauses 1 to 20, wherein at least about 8 antiviral binders are conjugated to the scaffold.

Clause 22: The biomolecule of any one of clauses 1 to 20, wherein at least about 12 antiviral binders are conjugated to the scaffold.

Clause 23: The biomolecule of any one of clauses 1 to 20, wherein at least about 24 antiviral binders are conjugated to the scaffold.

Clause 24: The biomolecule of any one of clauses 1 to 20, wherein at least about 36 antiviral binders are conjugated to the scaffold.

Clause 25: The biomolecule of any one of clauses 1 to 20, wherein about 60 antiviral binders are conjugated to the scaffold.

Clause 26: A composition comprising a plurality of the multivalent antiviral biomolecules of any one of clauses 1 to 25.

Clause 27: The composition of clause 26, further comprising a pharmaceutically acceptable adjuvant, excipient, and/or carrier, and wherein the composition is suitable for administration to a subject in need thereof.

Clause 28: A method of detecting a target virus using the multivalent antiviral biomolecule of any one of clauses 1 to 25.

Clause 290: A method of neutralizing a target virus using the multivalent antiviral biomolecule of any one of clauses 1 to 25.

Clause 30: A kit comprising the multivalent antiviral biomolecule of any one of clauses 1 to 25, and instructions for detecting or neutralizing a target virus.

1. A multivalent antiviral biomolecule comprising:
(i) a nucleic acid-based scaffold; and
(ii) a plurality of antiviral binders conjugated to the scaffold, wherein each antiviral binder of the plurality of binder targets one or more viral proteins, thereby neutralizing one or more viruses.

2. The biomolecule of claim 1, wherein the plurality of antiviral binders target the same viral protein.

3. The biomolecule of claim 1, wherein the plurality of antiviral binders target two or more distinct viral proteins.

4. The biomolecule of claim 1, wherein the nucleic acid-based scaffold comprises a DNA origami-based nanostructure.

5. The biomolecule of claim 1, wherein the nucleic acid-based scaffold comprises a DNA snub cube, wherein at least one edge of the DNA snub cube is conjugated to at least one antiviral binder of the plurality of antiviral binders.

6. The biomolecule of claim 5, wherein the DNA snub cube comprises 24 vertices and 60 edges.

7. (canceled)

8. The biomolecule of claim 1 wherein the plurality of antiviral binders are conjugated to the nucleic acid-based scaffold via a linker.

9. The biomolecule of claim 8, wherein the linker comprises a single-stranded DNA overhang coupled to the scaffold and a single-stranded DNA complementary to the single-stranded DNA overhang, wherein the complementary single-stranded DNA is conjugated to at least one antiviral binder of the plurality of antiviral binders.

10. (canceled)

11. (canceled)

12. The biomolecule of claim 11, wherein the complementary single-stranded DNA is conjugated to at least one antiviral binder of the plurality of antiviral binders via a SNAP-tag with a benzylguanine (BG) linker.

13. The biomolecule of claim 1, wherein at least one of the plurality of antiviral binders comprises a monoclonal antibody, a human antibody, a humanized antibody, a chimeric antibody, a single-domain antibody, an antibody fragment selected from the group consisting of Fab, Fab-C, Fab′-SH, Fv, scFv, and (Fab′)₂, an RNA aptamer, or a DNA aptamer.

14. (canceled)

15. (canceled)

16. (canceled)

17. (canceled)

18. The biomolecule of claim 13, wherein the aptamer comprises a docking domain to facilitate conjugation to the scaffold.

19. The biomolecule of claim 18, wherein the aptamer further comprises a linker region between the docking domain and a region of the aptamer that binds a viral protein.

20. The biomolecule of claim 19, wherein the linker region is from about 2 to about 40 base pairs in length.

21. The biomolecule of claim 1, wherein at least about 8 antiviral binders are conjugated to the scaffold.

22. (canceled)

23. The biomolecule of claim 1, wherein at least about 24 antiviral binders are conjugated to the scaffold.

24. The biomolecule of claim 1, wherein at least about 36 antiviral binders are conjugated to the scaffold.

25. The biomolecule of claim 1, wherein about 60 antiviral binders are conjugated to the scaffold.

26. A composition comprising a plurality of the multivalent antiviral biomolecules of claim 1.

27. (canceled)

28. A method of detecting or neutralizing a target virus, comprising using-contacting a sample or a subject with the multivalent antiviral biomolecule of claim 1.

29. (canceled)

30. A kit comprising the multivalent antiviral biomolecule of claim 1, and instructions for detecting or neutralizing a target virus.


SEQUENCE LISTING (205 sequences)

SEQ ID NO: 1 - DNA, 42 bp, Synthetic construct
  source 1..42: mol_type = other DNA
agactttttttcaaatatcgcggaagcaaacttttttccaac

SEQ ID NO: 2 - DNA, 35 bp, Synthetic construct
  source 1..35: mol_type = other DNA
aggtcgaaccatcaccttttcaaatcaagttcgga

SEQ ID NO: 3 - DNA, 51 bp, Synthetic construct
  source 1..51: mol_type = other DNA
acgtcattttaagggcgaaatgataagaggttttttcatttttgcgctgta

SEQ ID NO: 4 - DNA, 64 bp, Synthetic construct
  source 1..64: mol_type = other DNA
ttttttttttttttttttttttcactacgtaggattagagagtacctttaacagggcgatggcc

SEQ ID NO: 5 - DNA, 20 bp, Synthetic construct
  source 1..20: mol_type = other DNA
ttgctcctttaaccgtctat

SEQ ID NO: 6 - DNA, 36 bp, Synthetic construct
  source 1..36: mol_type = other DNA
tgattccattagatactttttatttcgcaaactcca

SEQ ID NO: 7 - DNA, 41 bp, Synthetic construct
  source 1..41: mol_type = other DNA
agtgttttttgttccagtttagtgccaagctttttttgcat

SEQ ID NO: 8 - DNA, 64 bp, Synthetic construct
  source 1..64: mol_type = other DNA
ttttttttttttttttttttttaacgtggatggtcaataacgacgttgtaatccactattaaag

SEQ ID NO: 9 - DNA, 20 bp, Synthetic construct
  source 1..20: mol_type = other DNA
aacgacggccggaacaagag

SEQ ID NO: 10 - DNA, 52 bp, Synthetic construct
  source 1..52: mol_type = other DNA
aattcctttttacacaacatatgcctaatgagttttttgagctaactggttg

SEQ ID NO: 11 - DNA, 41 bp, Synthetic construct
  source 1..41: mol_type = other DNA
cgaaatttttcggcaaaatcttccagtcgggtttttaaacc

SEQ ID NO: 12 - DNA, 64 bp, Synthetic construct
  source 1..64: mol_type = other DNA
ttttttttttttttttttttttcgagatagcacattaattgcgttgcgctccaaaagaatagcc

SEQ ID NO: 13 - DNA, 20 bp, Synthetic construct
  source 1..20: mol_type = other DNA
actgcccgctccttataaat

SEQ ID NO: 14 - DNA, 52 bp, Synthetic construct
  source 1..52: mol_type = other DNA
accctatttttaagggagcccgactattatagttttttcagaagcaaccgaa

SEQ ID NO: 15 - DNA, 35 bp, Synthetic construct
  source 1..35: mol_type = other DNA
ggcgaaagccgtaaagcactaaatttttggggtcg

SEQ ID NO: 16 - DNA, 52 bp, Synthetic construct
  source 1..52: mol_type = other DNA
gggcaatttttcagctgattgcaagcggtccatttttcgctggtttgggttc

SEQ ID NO: 17 - DNA, 49 bp, Synthetic construct
  source 1..49: mol_type = other DNA
ttttttttttttttttttttttaggtatcctgtttgatggtccccagca

SEQ ID NO: 18 - DNA, 52 bp, Synthetic construct
  source 1..52: mol_type = other DNA
tggatttttttatttacattgaaagggacatttttttctggccaacaagaat

SEQ ID NO: 19 - DNA, 42 bp, Synthetic construct
  source 1..42: mol_type = other DNA
acgtggtttttcacagacaatttacctttttttttttaatgg

SEQ ID NO: 20 - DNA, 48 bp, Synthetic construct
  source 1..48: mol_type = other DNA
ttttttttttttttttttttttacgtgacctgaaagcgtagagataga

SEQ ID NO: 21 - DNA, 36 bp, Synthetic construct
  source 1..36: mol_type = other DNA
acccttcagtagatttagtttgacccaattctgcga

SEQ ID NO: 22 - DNA, 42 bp, Synthetic construct
  source 1..42: mol_type = other DNA
gctcaatttttcatgttttaacattccatatatttttacagt

SEQ ID NO: 23 - DNA, 49 bp, Synthetic construct
  source 1..49: mol_type = other DNA
tttttttttttttttttttttttaccggtgtctggaagtttatatgcaa

SEQ ID NO: 24 - DNA, 35 bp, Synthetic construct
  source 1..35: mol_type = other DNA
ctaaagtacgaacgaaccaccagcgccattaaaaa

SEQ ID NO: 25 - DNA, 52 bp, Synthetic construct
  source 1..52: mol_type = other DNA
aaacattttttcaagaaaacagaactgatagctttttcctaaaacatcagaa

SEQ ID NO: 26 - DNA, 42 bp, Synthetic construct
  source 1..42: mol_type = other DNA
gataaatttttacagaggtgaatcgggagaaatttttcaata

SEQ ID NO: 27 - DNA, 34 bp, Synthetic construct
  source 1..34: mol_type = other DNA
aatctaaattgctgaatataatggatggcttaga

SEQ ID NO: 28 - DNA, 50 bp, Synthetic construct
  source 1..50: mol_type = other DNA
ttttttttttttttttttttttgcttaagcatcaccttgctgaaatgaaa

SEQ ID NO: 29 - DNA, 42 bp, Synthetic construct
  source 1..42: mol_type = other DNA
gctgagtttttagccagcagcaacctcaaatattttttcaaa

SEQ ID NO: 30 - DNA, 36 bp, Synthetic construct
  source 1..36: mol_type = other DNA
gcctggctcgaattcgttttttaatcatggtctcac

SEQ ID NO: 31 - DNA, 33 bp, Synthetic construct
  source 1..33: mol_type = other DNA
accagtcgatccccgggtaccgacaggtcgact

SEQ ID NO: 32 - DNA, 50 bp, Synthetic construct
  source 1..50: mol_type = other DNA
tttttttttttttttttttttttagagacacgaccagtaatagcagattc

SEQ ID NO: 33 - DNA, 35 bp, Synthetic construct
  source 1..35: mol_type = other DNA
gaacaatgtgaaattgttatccgcatagctgtttc

SEQ ID NO: 34 - DNA, 52 bp, Synthetic construct
  source 1..52: mol_type = other DNA
ctatattttttgtaaatgctgcaaactatcggtttttccttgctggttgcaa

SEQ ID NO: 35 - DNA, 42 bp, Synthetic construct
  source 1..42: mol_type = other DNA
caggaatttttaaacgctcatacatagcgatatttttgctta

SEQ ID NO: 36 - DNA, 49 bp, Synthetic construct
  source 1..49: mol_type = other DNA
ttttttttttttttttttttttctgtattaccgccagccataatatcca

SEQ ID NO: 37 - DNA, 36 bp, Synthetic construct
  source 1..36: mol_type = other DNA
ccatcacagtgtaaagcctggggcgagccggaagca

SEQ ID NO: 38 - DNA, 42 bp, Synthetic construct
  source 1..42: mol_type = other DNA
aggccatttttccgagtaaaatagcaatactttttttctttg

SEQ ID NO: 39 - DNA, 48 bp, Synthetic construct
  source 1..48: mol_type = other DNA
tttttttttttttttttttttttaagcaaattaaccgttggagtctgt

SEQ ID NO: 40 - DNA, 22 bp, Synthetic construct
  source 1..22: mol_type = other DNA
gtctttaccctccgatttagag

SEQ ID NO: 41 - DNA, 62 bp, Synthetic construct
  source 1..62: mol_type = other DNA
ttttttttttttttttttttttaagccggcggaccataaatcaaaaatcagcttgacgggga

SEQ ID NO: 42 - DNA, 42 bp, Synthetic construct
  source 1..42: mol_type = other DNA
ttcagatttttaaacgagaataacgtggcgagtttttaaagg

SEQ ID NO: 43 - DNA, 35 bp, Synthetic construct
  source 1..35: mol_type = other DNA
tacatttaagattaagaggaagcagcggattgcat

SEQ ID NO: 44 - DNA, 52 bp, Synthetic construct
  source 1..52: mol_type = other DNA
atataattttttcctgattgtactaatagatttttttagagccgtcattaga

SEQ ID NO: 45 - DNA, 42 bp, Synthetic construct
  source 1..42: mol_type = other DNA
ctttactttttaaacaattcgttattaatttttttttaaaag

SEQ ID NO: 46 - DNA, 49 bp, Synthetic construct
  source 1..49: mol_type = other DNA
ttttttttttttttttttttttcaaagaggatttagaagtaatagataa

SEQ ID NO: 47 - DNA, 48 bp, Synthetic construct
  source 1..48: mol_type = other DNA
tttttttttttttttttttttttccaaagcgaaccagaccgttttaat

SEQ ID NO: 48 - DNA, 36 bp, Synthetic construct
  source 1..36: mol_type = other DNA
tcgagcttagttggcaaatcaacaatcaatatctgg

SEQ ID NO: 49 - DNA, 36 bp, Synthetic construct
  source 1..36: mol_type = other DNA
ccctcagttgaaaggatttttattgaggaagagaac

SEQ ID NO: 50 - DNA, 36 bp, Synthetic construct
  source 1..36: mol_type = other DNA
tgtcgggggagaggcgtttttgtttgcgtatgagac

SEQ ID NO: 51 - DNA, 49 bp, Synthetic construct
  source 1..49: mol_type = other DNA
ttttttttttttttttttttttgaggaatcggccaacgcgctgccagct

SEQ ID NO: 52 - DNA, 35 bp, Synthetic construct
  source 1..35: mol_type = other DNA
gcattaataagtgtttttataatcgccagaatcct

SEQ ID NO: 53 - DNA, 52 bp, Synthetic construct
  source 1..52: mol_type = other DNA
ttgaaattttttaccgaccgtggattttagactttttaggaacggtacagtg

SEQ ID NO: 54 - DNA, 35 bp, Synthetic construct
  source 1..35: mol_type = other DNA
tgctttgttttcttttcaccagttgggcgccaggg

SEQ ID NO: 55 - DNA, 52 bp, Synthetic construct
  source 1..52: mol_type = other DNA
tttaactttttaacgccaacaatgcgccgctatttttcagggcgcgtgtgct

SEQ ID NO: 56 - DNA, 42 bp, Synthetic construct
  source 1..42: mol_type = other DNA
ttcctctttttgttagaatcaaccggaatcattttttaatta

SEQ ID NO: 57 - DNA, 49 bp, Synthetic construct
  source 1..49: mol_type = other DNA
tttttttttttttttttttttttggtacgagcacgtataacactatggt

SEQ ID NO: 58 - DNA, 35 bp, Synthetic construct
  source 1..35: mol_type = other DNA
gaaaggacctgagagagttgcagcccttcaccgcc

SEQ ID NO: 59 - DNA, 36 bp, Synthetic construct
  source 1..36: mol_type = other DNA
aagggctggcaagtgttttttagcggtcacgtaata

SEQ ID NO: 60 - DNA, 49 bp, Synthetic construct
  source 1..49: mol_type = other DNA
tttttttttttttttttttttttggcgcgggcgctagggcgaagaaagc

SEQ ID NO: 61 - DNA, 20 bp, Synthetic construct
  source 1..20: mol_type = other DNA
ttcatttgaaatttttgaat

SEQ ID NO: 62 - DNA, 64 bp, Synthetic construct
  source 1..64: mol_type = other DNA
tttttttttttttttttttttttttaacaatggctattagtctttaatgcgcaaattaattaca

SEQ ID NO: 63 - DNA, 36 bp, Synthetic construct
  source 1..36: mol_type = other DNA
aaacattgcttctgtatttttaatcgtcgcttgaaa

SEQ ID NO: 64 - DNA, 20 bp, Synthetic construct
  source 1..20: mol_type = other DNA
atccttgaaaggaaatacct

SEQ ID NO: 65 - DNA, 64 bp, Synthetic construct
  source 1..64: mol_type = other DNA
ttttttttttttttttttttttcccttagaacattttgacgctcaatcgtcattaattaatttt

SEQ ID NO: 66 - DNA, 20 bp, Synthetic construct
  source 1..20: mol_type = other DNA
aaagaacgcgtaataacatc

SEQ ID NO: 67 - DNA, 36 bp, Synthetic construct
  source 1..36: mol_type = other DNA
attagagaaaacttttttttttcaaatatatatggt

SEQ ID NO: 68 - DNA, 64 bp, Synthetic construct
  source 1..64: mol_type = other DNA
tttttttttttttttttttttttcgcaagacacttgcctgagtagaagaactatgcaaatccaa

SEQ ID NO: 69 - DNA, 64 bp, Synthetic construct
  source 1..64: mol_type = other DNA
ttttttttttttttttttttttcgttaaattaaacaggaggccgattaaaggtgataaataagg

SEQ ID NO: 70 - DNA, 20 bp, Synthetic construct
  source 1..20: mol_type = other DNA
aagaataaacgagcgggagc

SEQ ID NO: 71 - DNA, 20 bp, Synthetic construct
  source 1..20: mol_type = other DNA
ttcgagccagctgcgcgtaa

SEQ ID NO: 72 - DNA, 52 bp, Synthetic construct
  source 1..52: mol_type = other DNA
agagaattttttataaagtacccaatactgcgtttttgaatcgtcataacag

SEQ ID NO: 73 - DNA, 64 bp, Synthetic construct
  source 1..64: mol_type = other DNA
ttttttttttttttttttttttgaggcattccaccacacccgccgcgcttatgtaatttaggca

SEQ ID NO: 74 - DNA, 55 bp, Synthetic construct
  source 1..55: mol_type = other DNA
ttttttttttttttttttttttaatgctttaaaatattcattgaatctcctttgc

SEQ ID NO: 75 - DNA, 27 bp, Synthetic construct
  source 1..27: mol_type = other DNA
ccgaacgacaactcgtattaaacccct

SEQ ID NO: 76 - DNA, 20 bp, Synthetic construct
  source 1..20: mol_type = other DNA
tggaagggttgttatctaaa

SEQ ID NO: 77 - DNA, 52 bp, Synthetic construct
  source 1..52: mol_type = other DNA
ctaccattttttatcaaaattgtagattttcatttttggtttaacgtgccac

SEQ ID NO: 78 - DNA, 64 bp, Synthetic construct
  source 1..64: mol_type = other DNA
tttttttttttttttttttttttctgaataaatatctttaggagcactaacattggattatact

SEQ ID NO: 79 - DNA, 64 bp, Synthetic construct
  source 1..64: mol_type = other DNA
tttttttttttttttttttttttgcaacagtcagatgaatatacagtaacagattaacaccgcc

SEQ ID NO: 80 - DNA, 20 bp, Synthetic construct
  source 1..20: mol_type = other DNA
taccttttacggcggtcagt

SEQ ID NO: 81 - DNA, 52 bp, Synthetic construct
  source 1..52: mol_type = other DNA
aattgatttttgttaagcccaatagctatctttttttaccgaagcccagtta

SEQ ID NO: 82 - DNA, 42 bp, Synthetic construct
  source 1..42: mol_type = other DNA
ccagaatttttggaaaccgagaataggaaccctttttatgta

SEQ ID NO: 83 - DNA, 36 bp, Synthetic construct
  source 1..36: mol_type = other DNA
ccgtaaacgcctgtagtttttcattccacagacaag

SEQ ID NO: 84 - DNA, 42 bp, Synthetic construct
  source 1..42: mol_type = other DNA
taacattttttaaaaacagggaccctgaacaatttttagtca

SEQ ID NO: 85 - DNA, 36 bp, Synthetic construct
  source 1..36: mol_type = other DNA
gaggggttttgtcgtcttttttttccagacggctaa

SEQ ID NO: 86 - DNA, 52 bp, Synthetic construct
  source 1..52: mol_type = other DNA
acaacttttttttcaacagttagaaacgatttttttttttgtttaacgagaa

SEQ ID NO: 87 - DNA, 52 bp, Synthetic construct
  source 1..52: mol_type = other DNA
ttttattttttcctgaatctttaatttgccagtttttttacaaaataaagga

SEQ ID NO: 88 - DNA, 42 bp, Synthetic construct
  source 1..42: mol_type = other DNA
attgcgtttttaataataattctccaaaaggatttttgcctt

SEQ ID NO: 89 - DNA, 36 bp, Synthetic construct
  source 1..36: mol_type = other DNA
taattaatttcttaaatttttcagcttgatatacaa

SEQ ID NO: 90 - DNA, 52 bp, Synthetic construct
  source 1..52: mol_type = other DNA
aggtttttttttgaagccttatcgcccacgcattttttaaccgatataggcc

SEQ ID NO: 91 - DNA, 42 bp, Synthetic construct
  source 1..42: mol_type = other DNA
gctttttttttgcgggatcgtcatcgtaggaattttttcatt

SEQ ID NO: 92 - DNA, 52 bp, Synthetic construct
  source 1..52: mol_type = other DNA
aaaccatttttatcaataatcgtattaaaccatttttagtaccgcacgaggg

SEQ ID NO: 93 - DNA, 42 bp, Synthetic construct
  source 1..42: mol_type = other DNA
tagcaatttttcggctacagagaagtttccatttttttaaac

SEQ ID NO: 94 - DNA, 36 bp, Synthetic construct
  source 1..36: mol_type = other DNA
gggtacctaaaacgaatttttagaggcaaaatgtag

SEQ ID NO: 95 - DNA, 42 bp, Synthetic construct
  source 1..42: mol_type = other DNA
aataaatttttcaacatgttcaatagataagttttttcctga

SEQ ID NO: 96 - DNA, 36 bp, Synthetic construct
  source 1..36: mol_type = other DNA
acaagagcgattatactttttcaagcgcgaactgat

SEQ ID NO: 97 - DNA, 52 bp, Synthetic construct
  source 1..52: mol_type = other DNA
aaattgttttttgtcgaaatcaaagaagtttttttttgccagaggggacgac

SEQ ID NO: 98 - DNA, 42 bp, Synthetic construct
  source 1..42: mol_type = other DNA
cgtttatttttccagacgacggaggcgcagactttttggtca

SEQ ID NO: 99 - DNA, 36 bp, Synthetic construct
  source 1..36: mol_type = other DNA
atcatggacagatgaatttttcggtgtacagaagag

SEQ ID NO: 100 - DNA, 52 bp, Synthetic construct
  source 1..52: mol_type = other DNA
taatctttttttgacaagaactacataacgcctttttaaaaggaattaccct

SEQ ID NO: 101 - DNA, 36 bp, Synthetic construct
  source 1..36: mol_type = other DNA
aataatagaaagattctttttatcagttgagagctg

SEQ ID NO: 102 - DNA, 52 bp, Synthetic construct
  source 1..52: mol_type = other DNA
ctcatttttttcagtgaataagtagtaaattgtttttggcttgagattacct

SEQ ID NO: 103 - DNA, 42 bp, Synthetic construct
  source 1..42: mol_type = other DNA
tatgcgtttttattttaagaaaagaaaaatcttttttacgtt

SEQ ID NO: 104 - DNA, 64 bp, Synthetic construct
  source 1..64: mol_type = other DNA
ttttttttttttttttttttttctcagtacgcagcaccgtaatcagtagcgtagcggggttttg

SEQ ID NO: 105 - DNA, 41 bp, Synthetic construct
  source 1..41: mol_type = other DNA
aatgaatttttaccatcgatacaggcggataattttgtgcc

SEQ ID NO: 106 - DNA, 36 bp, Synthetic construct
  source 1..36: mol_type = other DNA
gtcgaacaaaagggcgtttttacattcaaccattat

SEQ ID NO: 107 - DNA, 52 bp, Synthetic construct
  source 1..52: mol_type = other DNA
tcattatttttaaggtgaatttagagccagcatttttaaatcaccagtcacc

SEQ ID NO: 108 - DNA, 64 bp, Synthetic construct
  source 1..64: mol_type = other DNA
ttttttttttttttttttttttagtatagcccggaataggagaaaattcatatggtttaccata

SEQ ID NO: 109 - DNA, 20 bp, Synthetic construct
  source 1..20: mol_type = other DNA
agcgccaaaggagggttgat

SEQ ID NO: 110 - DNA, 52 bp, Synthetic construct
  source 1..52: mol_type = other DNA
cataaatttttggtggcaacagtttattttgttttttcacaatcaattgtat

SEQ ID NO: 111 - DNA, 41 bp, Synthetic construct
  source 1..41: mol_type = other DNA
caccgtttttactcaggaggcagaaccgccatttttccctc

SEQ ID NO: 112 - DNA, 36 bp, Synthetic construct
  source 1..36: mol_type = other DNA
agagcactggcatgatttttttaagactcctacata

SEQ ID NO: 113 - DNA, 49 bp, Synthetic construct
  source 1..49: mol_type = other DNA
ttttttttttttttttttttttgcctcagaaccgccacccttttagtac

SEQ ID NO: 114 - DNA, 35 bp, Synthetic construct
  source 1..35: mol_type = other DNA
cgccaccccgtataaacagttaattgagtaacagt

SEQ ID NO: 115 - DNA, 22 bp, Synthetic construct
  source 1..22: mol_type = other DNA
tacccaaaagacaccaccctca

SEQ ID NO: 116 - DNA, 62 bp, Synthetic construct
  source 1..62: mol_type = other DNA
tttttttttttttttttttttttaacggaattttcagggatagcaagcccgaaacgcaataa

SEQ ID NO: 117 - DNA, 48 bp, Synthetic construct
  source 1..48: mol_type = other DNA
ttttttttttttttttttttttcagaccagtacaaactacacactgag

SEQ ID NO: 118 - DNA, 36 bp, Synthetic construct
  source 1..36: mol_type = other DNA
tttcgtcgagtgtactggtaatagcttttgatgata

SEQ ID NO: 119 - DNA, 20 bp, Synthetic construct
  source 1..20: mol_type = other DNA
gagataacccacagccctca

SEQ ID NO: 120 - DNA, 64 bp, Synthetic construct
  source 1..64: mol_type = other DNA
ttttttttttttttttttttttaatatcagatagttagcgtaacgatctaaataattgagcgct

SEQ ID NO: 121 - DNA, 35 bp, Synthetic construct
  source 1..35: mol_type = other DNA
aaagcgcatctgtatgggattttttagtaaatgaa

SEQ ID NO: 122 - DNA, 49 bp, Synthetic construct
  source 1..49: mol_type = other DNA
tttttttttttttttttttttttttgtctctgaatttaccgcagaatgg

SEQ ID NO: 123 - DNA, 20 bp, Synthetic construct
  source 1..20: mol_type = other DNA
aatccaaatatcagcggagt

SEQ ID NO: 124 - DNA, 64 bp, Synthetic construct
  source 1..64: mol_type = other DNA
tttttttttttttttttttttttttatcccgagaatagaaaggaacaactaaacagccatatta

SEQ ID NO: 125 - DNA, 49 bp, Synthetic construct
  source 1..49: mol_type = other DNA
ttttttttttttttttttttttttggtctccaaaaaaaaggttttcacg

SEQ ID NO: 126 - DNA, 35 bp, Synthetic construct
  source 1..35: mol_type = other DNA
ttgaaaaccttgatattcacaaacaggtcagacga

SEQ ID NO: 127 - DNA, 34 bp, Synthetic construct
  source 1..34: mol_type = other DNA
cgccaccccttgctttcgaggtggtatcggttta

SEQ ID NO: 128 - DNA, 50 bp, Synthetic construct
  source 1..50: mol_type = other DNA
tttttttttttttttttttttttcagtcagaaccgccaccctctcagagc

SEQ ID NO: 129 - DNA, 20 bp, Synthetic construct
  source 1..20: mol_type = other DNA
tgcacccagcccgatagttg

SEQ ID NO: 130 - DNA, 64 bp, Synthetic construct
  source 1..64: mol_type = other DNA
ttttttttttttttttttttttccgacaatgacaacaaccaaatcaagattagttgctatttcg

SEQ ID NO: 131 - DNA, 35 bp, Synthetic construct
  source 1..35: mol_type = other DNA
ataatcaacttgcagggagttaaattcggtcgctg

SEQ ID NO: 132 - DNA, 49 bp, Synthetic construct
  source 1..49: mol_type = other DNA
ttttttttttttttttttttttaggaatcaccggaaccagaatcttttc

SEQ ID NO: 133 - DNA, 20 bp, Synthetic construct
  source 1..20: mol_type = other DNA
tttttattttcaccctcagc

SEQ ID NO: 134 - DNA, 64 bp, Synthetic construct
  source 1..64: mol_type = other DNA
ttttttttttttttttttttttatcggaactcatcgagaacaagcaagccgagcgaaagacagc

SEQ ID NO: 135 - DNA, 35 bp, Synthetic construct
  source 1..35: mol_type = other DNA
ttttcatcagactttttcatgagggctttgaggac

SEQ ID NO: 136 - DNA, 49 bp, Synthetic construct
  source 1..49: mol_type = other DNA
tttttttttttttttttttttttaaggcattttcggtcatagtagcgcg

SEQ ID NO: 137 - DNA, 49 bp, Synthetic construct
  source 1..49: mol_type = other DNA
ttttttttttttttttttttttcattctacgaaggcaccaaaaatacgt

SEQ ID NO: 138 - DNA, 35 bp, Synthetic construct
  source 1..35: mol_type = other DNA
aatgccaagcaaggccggaaacgtagcaccattac

SEQ ID NO: 139 - DNA, 20 bp, Synthetic construct
  source 1..20: mol_type = other DNA
tttacgagcagaatacacta

SEQ ID NO: 140 - DNA, 64 bp, Synthetic construct
  source 1..64: mol_type = other DNA
ttttttttttttttttttttttccatcctaaaaacactcatctttgacccccaaaaataatatc

SEQ ID NO: 141 - DNA, 49 bp, Synthetic construct
  source 1..49: mol_type = other DNA
ttttttttttttttttttttttgactgatttgtatcatcgcacaaagta

SEQ ID NO: 142 - DNA, 35 bp, Synthetic construct
  source 1..35: mol_type = other DNA
caacggatgagccatttgggaatatcaccgtcacc

SEQ ID NO: 143 - DNA, 20 bp, Synthetic construct
  source 1..20: mol_type = other DNA
ggcttttgcacgcgacctgc

SEQ ID NO: 144 - DNA, 64 bp, Synthetic construct
  source 1..64: mol_type = other DNA
ttttttttttttttttttttttatagcgagatccatgttacttagccggaacataaaaaccaaa

SEQ ID NO: 145 - DNA, 36 bp, Synthetic construct
  source 1..36: mol_type = other DNA
gagggaagaccaactttgaaagaaagggaaccgaac

SEQ ID NO: 146 - DNA, 48 bp, Synthetic construct
  source 1..48: mol_type = other DNA
tttttttttttttttttttttttggtaaatattgacggaagattgagg

SEQ ID NO: 147 - DNA, 35 bp, Synthetic construct
  source 1..35: mol_type = other DNA
aaacgcaatggctgaccttcatcaccaggcgcata

SEQ ID NO: 148 - DNA, 49 bp, Synthetic construct
  source 1..49: mol_type = other DNA
ttttttttttttttttttttttggcagacaccacggaataatataaaag

SEQ ID NO: 149 - DNA, 20 bp, Synthetic construct
  source 1..20: mol_type = other DNA
ctaatgcagacggatattca

SEQ ID NO: 150 - DNA, 64 bp, Synthetic construct
  source 1..64: mol_type = other DNA
ttttttttttttttttttttttcgtaacaaatttaggaataccacattcaattacccaaatcaa

SEQ ID NO: 151 - DNA, 49 bp, Synthetic construct
  source 1..49: mol_type = other DNA
tttttttttttttttttttttttgttgaaacaccagaacgaggcttgcc

SEQ ID NO: 152 - DNA, 35 bp, Synthetic construct
  source 1..35: mol_type = other DNA
ctgacgaagcaaacgtagaaaattattacgcagta

SEQ ID NO: 153 - DNA, 52 bp, Synthetic construct
  source 1..52: mol_type = other DNA
tttttttttttttttttttttttgtgaatggtttaatttcaactgcagatag

SEQ ID NO: 154 - DNA, 32 bp, Synthetic construct
  source 1..32: mol_type = other DNA
ccgaacaatttttaagaaaagtaattaatcat

SEQ ID NO: 155 - DNA, 20 bp, Synthetic construct
  source 1..20: mol_type = other DNA
acagaatcaaggattaggat

SEQ ID NO: 156 - DNA, 36 bp, Synthetic construct
  source 1..36: mol_type = other DNA
gagaagtttgcctttatttttgcgtcagactgcccc

SEQ ID NO: 157 - DNA, 52 bp, Synthetic construct
  source 1..52: mol_type = other DNA
cttatttttttagcgtttgccgccaccaccggtttttaaccgcctcccagag

SEQ ID NO: 158 - DNA, 41 bp, Synthetic construct
  source 1..41: mol_type = other DNA
ccaccatttttccctcagagcggctgagactcttttctcaa

SEQ ID NO: 159 - DNA, 64 bp, Synthetic construct
  source 1..64: mol_type = other DNA
tttttttttttttttttttttttattaagacgccaccagaaccaccaccagtgaaacatgaaag

SEQ ID NO: 160 - DNA, 20 bp, Synthetic construct
  source 1..20: mol_type = other DNA
agccgccgcccctattattc

SEQ ID NO: 161 - DNA, 35 bp, Synthetic construct
  source 1..35: mol_type = other DNA
gtgcctgccccctgcctttttatttcggaaagcat

SEQ ID NO: 162 - DNA, 52 bp, Synthetic construct
  source 1..52: mol_type = other DNA
tgacagtttttgaggttgaggcaaataaatccttttttcattaaagcttcca

SEQ ID NO: 163 - DNA, 42 bp, Synthetic construct
  source 1..42: mol_type = other DNA
gtaagctttttgtcatacatgagttttaacggtttttggtca

SEQ ID NO: 164 - DNA, 49 bp, Synthetic construct
  source 1..49: mol_type = other DNA
ttttttttttttttttttttttaccgaaataaagaaattgcatttgcac

SEQ ID NO: 165 - DNA, 35 bp, Synthetic construct
  source 1..35: mol_type = other DNA
gtaaaacaagtcaggacgttgggctggctcattat

SEQ ID NO: 166 - DNA, 36 bp, Synthetic construct
  source 1..36: mol_type = other DNA
gattaaaaatcataggttttttctgagagactataa

SEQ ID NO: 167 - DNA, 35 bp, Synthetic construct
  source 1..35: mol_type = other DNA
gttttagggcttaggttgggttatacctttttaac

SEQ ID NO: 168 - DNA, 36 bp, Synthetic construct
  source 1..36: mol_type = other DNA
accgcgcttatccggttttttattctaagaagcggg

SEQ ID NO: 169 - DNA, 49 bp, Synthetic construct
  source 1..49: mol_type = other DNA
ttttttttttttttttttttttctcccgaacctcccgacttcgcgaggc

SEQ ID NO: 170 - DNA, 36 bp, Synthetic construct
  source 1..36: mol_type = other DNA
ctagaaattcttaccatttttgtataaagccccata

SEQ ID NO: 171 - DNA, 50 bp, Synthetic construct
  source 1..50: mol_type = other DNA
ttttttttttttttttttttttaacggcttaattgagaatcgaacgctca

SEQ ID NO: 172 - DNA, 34 bp, Synthetic construct
  source 1..34: mol_type = other DNA
acagtaggcgcctgtttatcaacagctaatgcag

SEQ ID NO: 173 - DNA, 36 bp, Synthetic construct
  source 1..36: mol_type = other DNA
tttgaaccagaaggagtttttcggaattatccatca

SEQ ID NO: 174 - DNA, 35 bp, Synthetic construct
  source 1..35: mol_type = other DNA
aacggaatcagatgatggcaattatcatattcctg

SEQ ID NO: 175 - DNA, 49 bp, Synthetic construct
  source 1..49: mol_type = other DNA
ttttttttttttttttttttttattacaacattattacaggaacgaact

SEQ ID NO: 176 - DNA, 36 bp, Synthetic construct
  source 1..36: mol_type = other DNA
acggaaatcgcgcagatttttggcgaattatgaaac

SEQ ID NO: 177 - DNA, 36 bp, Synthetic construct
  source 1..36: mol_type = other DNA
tagacggagcaaaagaagatgattcatttcaattac

SEQ ID NO: 178 - DNA, 48 bp, Synthetic construct
  source 1..48: mol_type = other DNA
ttttttttttttttttttttttctggagaattaactgaacaagcgcat

SEQ ID NO: 179 - DNA, 35 bp, Synthetic construct
  source 1..35: mol_type = other DNA
atcaatatagcagcctttacagagtcaaaaatgaa

SEQ ID NO: 180 - DNA, 49 bp, Synthetic construct
  source 1..49: mol_type = other DNA
ttttttttttttttttttttttaatatgtgagtgaataaccgtacataa

SEQ ID NO: 181 - DNA, 48 bp, Synthetic construct
  source 1..48: mol_type = other DNA
ttttttttttttttttttttttcattctgacctaaatttatttagtta

SEQ ID NO: 182 - DNA, 36 bp, Synthetic construct
  source 1..36: mol_type = other DNA
atttcatcaatcagatatagaaggcccaatagcaag

SEQ ID NO: 183 - DNA, 20 bp, Synthetic construct
  source 1..20: mol_type = other DNA
tggatagcgtcgacaaaagg

SEQ ID NO: 184 - DNA, 64 bp, Synthetic construct
  source 1..64: mol_type = other DNA
tttttttttttttttttttttttccagacggtaatagtaaaatgtttagactaaagtaattctg

SEQ ID NO: 185 - DNA, 34 bp, Synthetic construct
  source 1..34: mol_type = other DNA
atcattttagcaacactatcataacgaggcatag

SEQ ID NO: 186 - DNA, 50 bp, Synthetic construct
  source 1..50: mol_type = other DNA
tttttttttttttttttttttttaaggcggaacaaagaaaccgtaacatt

SEQ ID NO: 187 - DNA, 35 bp, Synthetic construct
  source 1..35: mol_type = other DNA
attgctttaacaatgaaatagcaataataagagca

SEQ ID NO: 188 - DNA, 49 bp, Synthetic construct
  source 1..49: mol_type = other DNA
ttttttttttttttttttttttagagaataccaagttacaattcgcctg

SEQ ID NO: 189 - DNA, 49 bp, Synthetic construct
  source 1..49: mol_type = other DNA
ttttttttttttttttttttttgagcaatagtgaatttatcagacgctg

SEQ ID NO: 190 - DNA, 35 bp, Synthetic construct
  source 1..35: mol_type = other DNA
agaagagtcgtctttccagagccaccaacgctaac

SEQ ID NO: 191 - DNA, 35 bp, Synthetic construct
  source 1..35: mol_type = other DNA
ttccttatcatatgcgttatacaaaaagcctgttt

SEQ ID NO: 192 - DNA, 49 bp, Synthetic construct
  source 1..49: mol_type = other DNA
ttttttttttttttttttttttagtatcattccaagaacggggctgtct

SEQ ID NO: 193 - DNA, 62 bp, Synthetic construct
  source 1..62: mol_type = other DNA
ttttttttttttttttttttcgagatagcacattaattgcgttgcgctccaaaagaatagcc

SEQ ID NO: 194 - DNA, 62 bp, Synthetic construct
  source 1..62: mol_type = other DNA
ttttttttttttttttttttcactacgtaggattagagagtacctttaacagggcgatggcc

SEQ ID NO: 195 - DNA, 62 bp, Synthetic construct; identical to SEQ ID NO: 193
  source 1..62: mol_type = other DNA

SEQ ID NO: 196 - DNA, 53 bp, Synthetic construct
  source 1..53: mol_type = other DNA
ttttttttttttttttttttaatgctttaaaatattcattgaatctcctttgc

SEQ ID NO: 197 - DNA, 62 bp, Synthetic construct
  source 1..62: mol_type = other DNA
tttttttttttttttttttttgcaacagtcagatgaatatacagtaacagattaacaccgcc

SEQ ID NO: 198 - DNA, 62 bp, Synthetic construct
  source 1..62: mol_type = other DNA
ttttttttttttttttttttctcagtacgcagcaccgtaatcagtagcgtagcggggttttg

SEQ ID NO: 199 - DNA, 47 bp, Synthetic construct
  source 1..47: mol_type = other DNA
ttttttttttttttttttttgcctcagaaccgccacccttttagtac

SEQ ID NO: 200 - DNA, 62 bp, Synthetic construct
  source 1..62: mol_type = other DNA
ttttttttttttttttttttatagcgagatccatgttacttagccggaacataaaaaccaaa

SEQ ID NO: 201 - DNA, 50 bp, Synthetic construct
  source 1..50: mol_type = other DNA
tttttttttttttttttttttgtgaatggtttaatttcaactgcagatag

SEQ ID NO: 202 - DNA, 47 bp, Synthetic construct
  source 1..47: mol_type = other DNA
ttttttttttttttttttttaatatgtgagtgaataaccgtacataa

SEQ ID NO: 203 - DNA, 46 bp, Synthetic construct
  source 1..46: mol_type = other DNA
ttttttttttttttttttttcattctgacctaaatttatttagtta

SEQ ID NO: 204 - DNA, 47 bp, Synthetic construct
  source 1..47: mol_type = other DNA
ttttttttttttttttttttgagcaatagtgaatttatcagacgctg

SEQ ID NO: 205 - DNA, 47 bp, Synthetic construct
  source 1..47: mol_type = other DNA
ttttttttttttttttttttagtatcattccaagaacggggctgtct